(19) United States
(12) Patent Application Publication
(10) Pub. No.: US 2024/0282408 A1
(43) Pub. Date: Aug. 22, 2024

(54) Method For Predicting Protein Binding Site

(71) Applicant: DEARGEN INC., Daejeon (KR)

(72) Inventors: Daeseok LEE, Seoul (KR); Jeunghyun BYUN, Seoul (KR); Bonggun SHIN, Gyeongsangbuk-do (KR)

(73) Assignee: DEARGEN INC., Daejeon (KR)

(21) Appl. No.: 18/497,318

(22) Filed: Oct. 30, 2023

Related U.S. Application Data
(60) Provisional application No. 63/446,242, filed on Feb. 16, 2023.

(51) Int. Cl. G16B 20/30; G16B 40/20

(52) U.S. Cl. CPC G16B 20/30; G16B 40/20

(57) ABSTRACT

Disclosed is a method for predicting a binding site of a protein, the method performed by one or more processors of a computing device.

The method may include: obtaining one or more candidate data; filtering the one or more candidate data, and obtaining the filtered candidate data, by using a first neural network model for detecting a binding site; and predicting a binding residue based on the filtered candidate data by using a second neural network model for identifying the binding residue, and the first neural network model may share some parameters with the second neural network model.

### TECHNICAL FIELD

The present disclosure relates to a method for predicting a binding site of a protein, and more particularly, to a method which uses a first neural network model for detecting a binding site for predicting a binding site and a second neural network model for identifying a binding residue, which are two neural network models sharing some parameters to predict the binding site and predict the binding residue for the predicted binding site.

### BACKGROUND ART

It was a very important task in many virtual and actual drug discovery scenarios to determine small molecule binding sites of target proteins in the related art. Therefore, it is not always easy to find such a binding position based on domain knowledge or traditional methods, so recently, various deep learning methods have been developed to predict the binding position in a protein structure. In this regard, the recent structure-based deep learning method is primarily based only on 3D CNN architecture, which operates in a grid-shaped input. However, a layer that performs a convolutional task in such a deep learning method could have difficulty due to a “long-term dependency” problem. Specifically, in the convolution-based method, a task of a convolutional layer is applied only locally, so a deep convolutional architecture structure had to be applied so as to take a large accommodation field so as for a neuron to capture a global pattern. As a result, the long-term dependency problem occurs and there is a problem in that training is interfered.

In this regard, there is a need for a method for predicting the binding site more accurately using the neural network model and predicting the binding residue for the predicted binding site.

On the other hand, the present disclosure has been derived at least based on the technical background described above, but the technical problem or object of the present disclosure is not limited to solving the problems or disadvantages described above. That is, the present disclosure may cover various technical issues related to the content to be described below, in addition to the technical issues discussed above.

Meanwhile, Chinese Patent Publication No. 115954050 (Apr. 11, 2023) relates to a deep learning model and a prediction method based on an integrated sequence and a structural feature of a protein process.

### SUMMARY OF THE INVENTION

The present disclosure has been made in an effort to provide a method for predicting a binding site of a protein, and more particularly, to predicting, by using a first neural network model for detecting a binding site for predicting a binding site and a second neural network model for identifying a binding residue, which are two neural network models sharing some parameters, the binding site and predicting the binding residue for the predicted binding site.

Meanwhile, a technical object to be achieved by the present disclosure is not limited to the above-mentioned technical object, and various technical objects can be included within the scope which is apparent to those skilled in the art from contents to be described below.

An exemplary embodiment of the present disclosure provides a method performed by a computing device. The method may include: obtaining one or more candidate data; filtering the one or more candidate data, and obtaining the filtered candidate data, by using a first neural network model for detecting a binding site; and predicting a binding residue based on the filtered candidate data by using a second neural network model for identifying the binding residue, and the first neural network model may share some parameters with the second neural network model.

Alternatively, the one or more candidate data may include one or more candidate binding sites of a protein, or a center of each of the candidate binding sites, and the obtaining of the one or more candidate data may include obtaining the one or more candidate binding sites of a protein, or the center of each of the candidate binding sites by using an algorithm for predicting the binding site in a protein structure. Alternatively, the first neural network model may include a first sub neural network for extracting a local feature of the binding site, and a second sub neural network for globally aggregating the local features, and the second neural network model may share at least one of the first sub neural network or the second sub neural network with the first neural network model.

Alternatively, the first sub neural network for extracting the local feature of the binding site may include a 3D convolutional network, the second sub neural network for globally aggregating the local features may include a geometric attention layer, and the first neural network model may further include a third sub neural network for mapping the feature aggregated through the second sub neural network to a single scalar quantity.

Alternatively, the first sub neural network may be capable of performing an operation of applying a grid alignment.

Alternatively, the second sub neural network may perform an operation of randomly transforming an orientation of a residue in a training process of at least one of the first neural network or the second neural network.

Alternatively, the filtering of the one or more candidate data, and obtaining the filtered candidate data, by using the first neural network model for detecting a binding site may include calculating a druggability score for each of the one or more candidate data by using the first neural network model for detecting the binding site, and obtaining the filtered candidate data based on the druggability score for each of the candidate data.

Alternatively, the first neural network model may be a model trained based on an operation of obtaining first training data and first ground truth data corresponding to the first training data, an operation of predicting a training binding site based on the first training data using the first neural network model, and an operation of training the first neural network model based on the predicted training binding site and the first ground truth data, and the second neural network model may be a model trained based on an operation of obtaining second training data and second ground truth data corresponding to the second training data, an operation of predicting a training binding residue based on the one or more training data using the second neural network model, and an operation of training the second neural network model based on the predicted training binding residue and the second ground truth data.

Alternatively, the first neural network model may be a model trained based on an operation of performing training for the first neural network model after performing the training process of the second neural network model.

Alternatively, the first neural network model may share some parameters with the trained second neural network model, and the operation of performing training for the first neural network model after performing the training process of the second neural network model may be a model trained based on an operation of setting some parameters shared with the trained second neural network model as an initial condition, and an operation of performing the training for the first neural network model based on the set initial condition.

Another exemplary embodiment of the present disclosure provides a method performed by a computing device. The method may include: obtaining training data, ground truth data corresponding to the training data, and external data; aligning the training data and the external data, and obtaining aligned external data corresponding to the training data; and assigning first sub data of the training data as sub data of the aligned external data based on the ground truth data to obtain augmented data, and the augmented data may be used in the process of training at least one of the first neural network model for detecting the binding site or the second neural network model for identifying the binding residue.

Alternatively, the aligning of the training data and the external data, and obtaining of the aligned external data corresponding to the training data may include obtaining an amino acid sequence associated with the training data, and when the obtained amino acid sequence is conserved in the aligned external data with the training data, obtaining aligned external data corresponding to the training data.

Alternatively, the training data may include a training protein structure, the ground truth data includes one or more ground truth binding sites for the training protein structure and a center of each of the ground truth binding sites, and the assigning the first sub data of the training data as the sub data of the aligned external data based on the ground truth data to obtain the augmented data may include assigning the center of the ground truth binding site to a center of a binding site of the aligned external data to obtain first augmented data.

Alternatively, the assigning the center of the ground truth binding site to the center of the binding site of the aligned external data corresponding to the training data to obtain the first augmented data may include obtaining a center of a candidate binding site by using an algorithm for predicting the binding site in the protein structure for the external data, obtaining the center of the binding site of the aligned external data corresponding to the training data, measuring a distance between the obtained center of the candidate binding site of the aligned external data and the center of the binding site corresponding to the training data, and when the measured distance is within a predetermined threshold, assigning the center of the ground truth binding site to the center of the binding site of the aligned external data corresponding to the training data to obtain the first augmented data.

Alternatively, the ground truth data may further include a ground truth binding residue for each of the ground truth binding sites, and the assigning the center of the ground truth binding site to the center of the binding site of the aligned external data corresponding to the training data to obtain the first augmented data may include calculating a ratio at which the binding residue of the aligned external data corresponding to the training data corresponds to the ground truth binding residue, and when the calculated ratio is equal to or more than a predetermined threshold, assigning the center of the ground truth binding site to the center of the binding site of the aligned external data corresponding to the training data to obtain the first augmented data.

Alternatively, the training data may include a first training protein structure, the ground truth data may include a first ground truth biding residue for the first training protein structure, and the assigning the first sub data of the training data as the sub data of the aligned external data based on the ground truth data to obtain the augmented data may include assigning the first ground truth binding residue to the binding residue of the aligned external data corresponding to the training data to obtain second augmented data.

Alternatively, the training data may further include a second training protein structure, the ground truth data may further include a second ground truth biding residue for the second training protein structure, and the assigning the first ground truth binding residue to the binding residue of the aligned external data corresponding to the training data to obtain the second augmented data may include assigning the first ground truth binding residue to a first binding residue of the aligned external data corresponding to the training data to obtain a first assigned binding residue, assigning the second ground truth binding residue to a second binding residue of the aligned external data corresponding to the training data to obtain a second assigned binding residue, and obtaining the second augmented data based on the first assigned binding residue and the second assigned binding residue.

Still another exemplary embodiment of the present disclosure provides a computer program stored in a computer-readable storage medium. The computer program may cause one or more processors to perform operations for predicting a binding site of a protein when the computer program is executed by one or more processors, and the operations may include: an operation of obtaining one or more candidate data; an operation of filtering the one or more candidate data, and obtaining the filtered candidate data, by using a first neural network model for detecting a binding site; and an operation of predicting a binding residue based on the filtered candidate data by using a second neural network model for identifying the binding residue, and the first neural network model may share some parameters with the second neural network model.

Alternatively, the operation of filtering the one or more candidate data, and obtaining the filtered candidate data, by using the first neural network model for detecting a binding site may include an operation of calculating a druggability score for each of the one or more candidate data by using the first neural network model for detecting the binding site, and an operation of obtaining the filtered candidate data based on the druggability score for each of the candidate data.

Yet another exemplary embodiment of the present disclosure provides a computing device. The device may be configured to obtain one or more candidate data, filter the one or more candidate data, and obtain the filtered candidate data, by using a first neural network model for detecting a binding site, and predict a binding residue based on the filtered candidate data by using a second neural network model for identifying the binding residue, and the first neural network model may share some parameters with the second neural network model.

Still yet another exemplary embodiment of the present disclosure provides a data structure included in a computer-readable storage medium. The data structure may correspond to a parameter of a neural network, and the neural network performs the following steps at least partially based on the parameter, and the steps may include: obtaining one or more candidate data; filtering the one or more candidate data, and obtaining the filtered candidate data, by using a first neural network model for detecting a binding site; and predicting a binding residue based on the filtered candidate data by using a second neural network model for identifying the binding residue, and the first neural network model may share some parameters with the second neural network model.

According to an exemplary embodiment of the present disclosure relates to a method for predicting a binding site of protein, and more particularly, a first neural network model for detecting a binding site for predicting a binding site and a second neural network model for identifying a binding residue are used, which are two neural network models sharing some parameters to predict the binding site and predict the binding residue for the predicted binding site, thereby more accurately predicting a binding pocket (a binding residue and a binding site) of a protein.

According to an exemplary embodiment of the present disclosure, by augmenting training data based on homology of the protein, overfitting of a model can be prevented.

Meanwhile, the effects of the present disclosure are not limited to the above-mentioned effects, and various effects can be included within the scope which is apparent to those skilled in the art from contents to be described below.

### BRIEF DESCRIPTION OF THE DRAWINGS

FIG. 1 is a block diagram of a computing device for predicting a binding site of a protein according to an exemplary embodiment of the present disclosure.

FIG. 2 is a schematic view illustrating a network function according to an exemplary embodiment of the present disclosure.

FIG. 3 is a flowchart illustrating a method for predicting a binding site of a protein according to an exemplary embodiment of the present disclosure.

FIG. 4 is a schematic view illustrating a process for predicting a binding site of a protein according to an exemplary embodiment of the present disclosure.

FIG. 5A is a schematic view for describing a process of training a second neural network model for identifying a binding residue according to an exemplary embodiment of the present disclosure.

FIG. 5B is a schematic view for describing a process of training a first neural network model for detecting a binding site, which shares some parameters with a second neural network model according to an exemplary embodiment of the present disclosure.

FIG. 6 is a flowchart illustrating a method for obtaining augmented data for training at least one of a first neural network model for detecting a binding site or a second neural network model for identifying a binding residue according to an exemplary embodiment of the present disclosure.

FIG. 7A is a schematic view for describing a process of obtaining first according to an exemplary embodiment of the present disclosure.

FIG. 7B is a schematic view for describing a process of obtaining second augmented data for training a second neural network model for identifying a binding residue according to an exemplary embodiment of the present disclosure.

FIG. 8 is a schematic view for comparing and describing performing transfer learning and not performing the transfer learning when training the first neural network model according to an exemplary embodiment of the present disclosure.

FIG. 9 is a schematic view for comparing and describing performing data augmentation and not performing the data augmentation when training the first and second neural network models according to an exemplary embodiment of the present disclosure.

FIG. 10 is a schematic view illustrating a lower domain of human serum albumin (HAS) according to an exemplary embodiment of the present disclosure.

FIGS. 11A-11B are schematic views for comparing and describing a prediction result according to an exemplary embodiment of the present disclosure and a prediction result of existing methods.

FIG. 12 is a normal and schematic view of an exemplary computing environment in which the exemplary embodiments of the present disclosure may be implemented.

### DETAILED DESCRIPTION

Various exemplary embodiments will now be described with reference to drawings. In the present specification, various descriptions are presented to provide appreciation of the present disclosure. However, it is apparent that the exemplary embodiments can be executed without the specific description.

“Component”, “module”, “system”, and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers.

Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “at least one of A or B” should be interpreted to mean “a case including only A”, “a case including only B”, and “a case in which A and B are combined”.

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally In terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented exemplary embodiments is provided so that those skilled in the art of the present disclosure use or implement the present disclosure. Various modifications to the exemplary embodiments will be apparent to those skilled in the art. Generic principles defined herein may be applied to other embodiments without departing from the scope of the present disclosure. Therefore, the present disclosure is not limited to the exemplary embodiments presented herein. The present disclosure should be analyzed within the widest range which is coherent with the principles and new features presented herein.

In the present disclosure, a network function and an artificial neural network and a neural network may be interchangeably used.

FIG. 1 is a block diagram of a computing device for predicting a binding site of a protein according to an exemplary embodiment of the present disclosure.

A configuration of the computing device 100 illustrated in FIG. 1 is only an example shown through simplification. In an exemplary embodiment of the present disclosure, the computing device 100 may include other components for performing a computing environment of the computing device 100 and only some of the disclosed components may constitute the computing device 100.

The computing device 100 may include a processor 110, a memory 130, and a network unit 150.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for training the neural network. The processor (110) is capable of performing computations for deep learning (DL), including processing input data for training, extracting features from input data, calculating errors, and updating the weights of neural network models using backpropagation. At least one of the CPU, GPGPU, and TPU of the processor 110 may process training of a network function. For example, both the CPU and the GPGPU may process the training of the network function and data classification using the network function. Further, in an exemplary embodiment of the present disclosure, processors of a plurality of computing devices may be used together to process the training of the network function and the data classification using the network function. Further, the computer program executed in the computing device according to an exemplary embodiment of the present disclosure may be a CPU, GPGPU, or TPU executable program.

According to an exemplary embodiment of the present disclosure, the memory 130 may store any type of information generated or determined by the processor 110 and any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit 150 according to an exemplary embodiment of the present disclosure may use various wired communication systems such as public switched telephone network (PSTN), x digital subscriber line (xDSL), rate adaptive DSL (RADSL), multi rate DSL (MDSL), very high speed DSL (VDSL), universal asymmetric DSL (UADSL), high bit rate DSL (HDSL), and local area network (LAN).

The network unit 150 presented in the present disclosure may use various wireless communication systems such as code division multi access (CDMA), time division multi access (TDMA), frequency division multi access (FDMA), orthogonal frequency division multi access (OFDMA), single carrier-FDMA (SC-FDMA), and other systems.

In the present disclosure, the network unit 150 may be configured regardless of communication modes such as wired and wireless modes and constituted by various communication networks including a personal area network (PAN), a wide area network (WAN), and the like. Further, the network may be known World Wide Web (WWW) and may adopt a wireless transmission technology used for short-distance communication, such as infrared data association (IrDA) or Bluetooth. The techniques described in the present disclosure may also be used in other networks mentioned above.

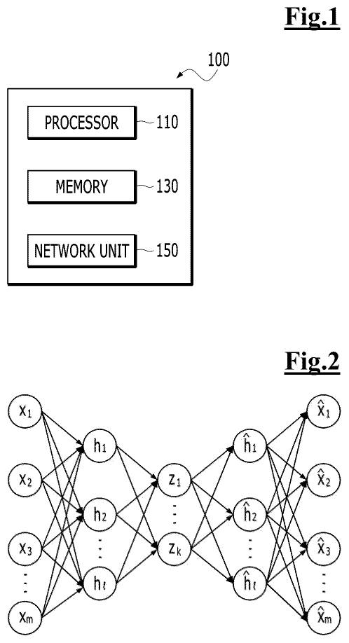

FIG. 2 is a conceptual view illustrating a neural network according to an exemplary embodiment of the present disclosure.

Throughout the present specification, a computation model, the neural network, a network function, and the neural network may be used as the same meaning. The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the link may relatively form the relationship between an input node and an output node. Concepts of the input node and the output node are relative and a predetermined node which has the output node relationship with respect to one node may have the input node relationship in the relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and the weight is variable by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights granted to the respective links in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, a definition of the layer is predetermined for description and the order of the layer in the neural network may be defined by a method different from the aforementioned method. For example, the layers of the nodes may be defined by the distance from a final output node.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean nodes constituting the neural network other than the initial input node and the final output node.

In the neural network according to an exemplary embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another exemplary embodiment of the present disclosure, the number of nodes of the input layer may be smaller than the number of nodes of the output layer, and the neural network may be a neural network of a type in which the number of nodes decreases from the input layer to the hidden layer. Further, in the neural network according to yet another exemplary embodiment of the present disclosure, the number of nodes of the input layer may be larger than the number of nodes of the output layer, and the neural network may be a neural network of a type in which the number of nodes increases from the input layer to the hidden layer. The neural network according to still yet another exemplary embodiment of the present disclosure may be a neural network of a type in which the neural networks are combined.

A deep neural network (DNN) may refer to a neural network that includes a plurality of hidden layers in addition to the input and output layers. When the deep neural network is used, the latent structures of data may be determined. That is, latent structures of photos, text, video, voice, and music (e.g., what objects are in the photo, what the content and feelings of the text are, what the content and feelings of the voice are) may be determined. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siam network, a Generative Adversarial Network (GAN), and the like. The description of the deep neural network described above is just an example and the present disclosure is not limited thereto.

In an exemplary embodiment of the present disclosure, the network function may include the auto encoder. The auto encoder may be a kind of artificial neural network for outputting output data similar to input data. The auto encoder may include at least one hidden layer and odd hidden layers may be disposed between the input and output layers. The number of nodes in each layer may be reduced from the number of nodes in the input layer to an intermediate layer called a bottleneck layer (encoding), and then expanded symmetrical to reduction to the output layer (symmetrical to the input layer) in the bottleneck layer. The auto encoder may perform non-linear dimensional reduction. The number of input and output layers may correspond to a dimension after preprocessing the input data. The auto encoder structure may have a structure in which the number of nodes in the hidden layer included in the encoder decreases as a distance from the input layer increases. When the number of nodes in the bottleneck layer (a layer having a smallest number of nodes positioned between an encoder and a decoder) is too small, a sufficient amount of information may not be delivered, and as a result, the number of nodes in the bottleneck layer may be maintained to be a specific number or more (e.g., half of the input layers or more).

The neural network may be trained in at least one scheme of supervised learning, unsupervised learning, semi supervised learning, or reinforcement learning. The learning of the neural network may be a process in which the neural network applies knowledge for performing a specific operation to the neural network.

The neural network may be trained in a direction to minimize errors of an output. The training of the neural network is a process of repeatedly inputting training data into the neural network and calculating the output of the neural network for the training data and the error of a target and back-propagating the errors of the neural network from the output layer of the neural network toward the input layer in a direction to reduce the errors to update the weight of each node of the neural network. In the case of the supervised learning, the training data labeled with a correct answer is used for each training data (i.e., the labeled training data) and in the case of the unsupervised learning, the correct answer may not be labeled in each training data. That is, for example, the training data in the case of the supervised learning related to the data classification may be data in which category is labeled in each training data. The labeled training data is input to the neural network, and the error may be calculated by comparing the output (category) of the neural network with the label of the training data. As another example, in the case of the unsupervised learning related to the data classification, the training data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a training cycle (epoch). The learning rate may be applied differently according to the number of repetition times of the training cycle of the neural network. For example, in an initial stage of the training of the neural network, the neural network ensures a certain level of performance quickly by using a high learning rate, thereby increasing efficiency and uses a low learning rate in a latter stage of the training, thereby increasing accuracy.

In training of the neural network, the training data may be generally a subset of actual data (i.e., data to be processed using the trained neural network), and as a result, there may be a training cycle in which errors for the training data decrease, but the errors for the actual data increase. Overfitting is a phenomenon in which the errors for the actual data increase due to excessive training of the training data. For example, a phenomenon in which the neural network that trains a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the training data, regularization, dropout of omitting a part of the node of the network in the process of training, utilization of a batch normalization layer, etc., may be applied.

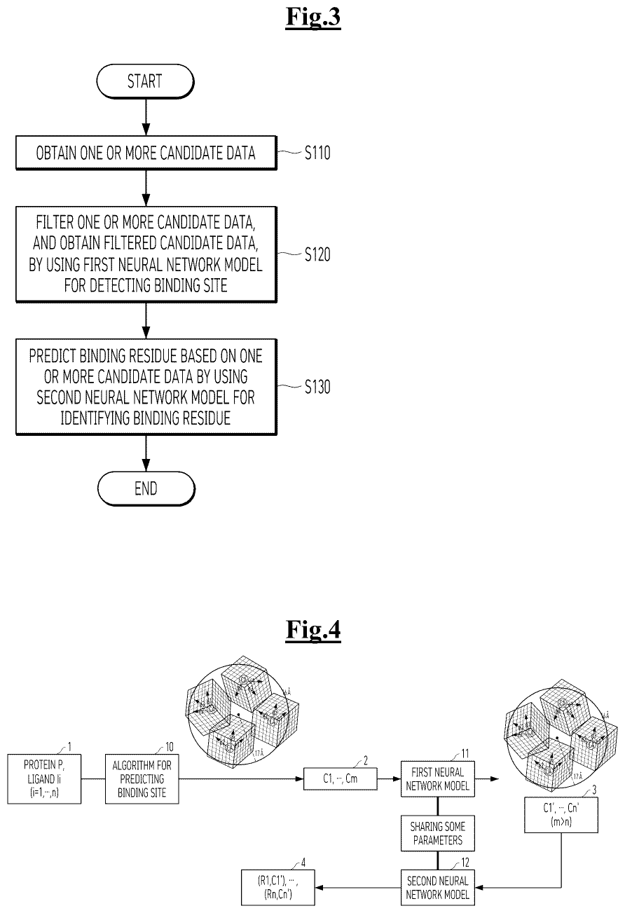

FIG. 3 is a flowchart illustrating a method for predicting a binding site of a protein according to an exemplary embodiment of the present disclosure.

A computing device 100 according to an exemplary embodiment of the present disclosure may directly obtain “information for predicting a binding site of a protein” or receive the “information for predicting a binding site of a protein” from an external system. The external system may be a server or database that stores and manages the information for predicting a binding site of a protein. The computing device 100 may use the information obtained directly or received from the external system as “input data for predicting a binding site of a protein”.

According to an exemplary embodiment of the present disclosure, the computing device 100 may obtain one or more candidate data (S110). In this case, one or more candidate data may include one or more candidate binding sites of the protein, and a center of each of the candidate binding sites. Specifically, the computing device 100 may obtain one or more candidate binding sites and the center of each of the candidate binding sites in the protein using an algorithm (model) for predicting the binding site in a protein structure. In this case, as an example of the algorithm, an algorithm for analyzing biological information related to the protein structure, such as Fpocket, and detecting a pocket (binding residue and binding site) of the protein may be used, but the present disclosure is not limited thereto, and various examples may be used. Specifically, the computing device 100 may find heavy atoms S1, . . . , Sm corresponding to an area which is likely to geometrically become the binding pocket (binding residue and binding site) on the surface of the protein structure through the algorithm, and obtain C1, . . . , Cm which are centers of the respective candidate binding sites by taking a center of an atomic mass for each of S1 to Sm. More specifically, the computing device 100 approximates a local curvature to a radius of an alpha sphere which is a “sphere having four heavy atoms, but having no heavy atom therein” by using the algorithm to obtain one or more candidate binding sites of the protein and the center of each of the candidate binding sites. Through this, the computing device 100 may obtain the one or more candidate data including one or more candidate binding sites of the protein and the center of each of the candidate binding sites by using the algorithm. Meanwhile, the obtained one or more candidate data may be used in the process of predicting the binding residue to be described below, and hereinafter, a specific process thereof will be described below through FIG. 4.

According to an exemplary embodiment of the present disclosure, the computing device 100 uses the first neural network model for detecting the binding site to filter one or more candidate data obtained through step S110, and obtain the filtered candidate data (S120). In this case, the first neural network model for detecting the binding site may mean a model which performs an operation of identifying the binding site which is a position bound with ligand (drug or other molecules) in a given protein. Meanwhile, the first neural network model may share some parameters with the second neural network model to be described below. In this case, two neural network models sharing some parameters may mean that two models use several weights or parameters similarly, and the shared parameter may be used for transfer learning that trains one neural network model, and then uses some trained weights of the model for other related tasks, and may be used for hybrid models which combine and use two or more other model architectures, but the present disclosure is not limited thereto. Through this, the computing device 100 may reduce a size of the model and more effectively use the training data, and enhance efficiency of the model and reduce overfitting. Further, the first neural network model may include a first sub neural network for extracting a local feature of the binding site and a second sub neural network for globally aggregating the local features. For example, the first sub neural network for extracting the local feature of the binding site may include a 3D convolutional neural network (CNN), and the second sub neural network for globally aggregating the local features may include a geometric attention layer. For example, the second sub neural network may be constituted by a plurality of geometric self-attention layers. Further, the first neural network model may further include a third sub neural network for mapping the feature aggregated through the second sub neural network to a single scalar quantity, and the third sub neural network may be configured through a point-wise feed-forward layer and a mean-reduction operation. In this case, the single scalar quantity may represent an amount expressed by one number or value.

The computing device 100 uses the first neural network model for detecting the binding site to calculate a druggability score for each of the one or more candidate data, and obtain the filtered candidate data based on the druggability score for each of the candidate data. For example, the computing device 100 may calculate the druggability score in specific Ci (i=1, . . . , m) by using the protein and the candidate binding site center C1, . . . , Cm as an input of the first neural network model, and filters n candidate binding site centers among the candidate binding site centers C1, . . . , Cm in the order of a higher calculated druggability score to obtain C1′, . . . , Cn′ which are filtered candidate binding site centers. In this case, the center of the candidate binding site may include a center of mass of atoms, for example. In this process, the computing device 100 may perform an operation of applying a grid alignment through the first sub neural network, and transform a surrounding of Ci′ (i=1, . . . , n) into an amino acid unit 3D grid set, and process the grids through the first neural network model and generate an output. In this case, even though rotation is applied to an input through a process of aligning the grids, SE-(3) immutability may be satisfied so that the output is not changed, and the SE-(3) immutability may mean conversion immutability in this space after a Euclidean space is extended to an SE(3) space which is an extended space. In this regard, since information such as the binding site of the protein structure is not changed and should be maintained regardless of a reference frame, additional processes such as using an axis aligned according to a orientation of the residue without using an arbitrary xyz axis when configuring the grid may be performed so that the SE-(3) immutability is satisfied in exemplary embodiment of the present disclosure. Meanwhile, the 3D grid may mean that the space is divided into the grid, and each grid cell may have a predetermined size and a predetermined shape, and represent a position in a 3D space, and the computing device 100 may express a specific local environment of the protein by using the amino acid unit 3D grid set with respect to one or more candidate data. Additionally, the computing device 100 may perform an operation of randomly transforming the orientation of the residue through the second sub neural network in the training process of at least one of the first neural network or the second neural network. In this regard, the computing device 100 may increase diversity of geometrical information without fully forgetting an original residue orientation through random conversion for the training data in a training process to be described below, and accordingly obtain an effect of augmenting the training data. Meanwhile, the filtered candidate data may be used in a process of predicting the binding residue by using the second neural network model for identifying the binding residue to be described below, and hereinafter, a specific process thereof will be described below through FIG. 4.

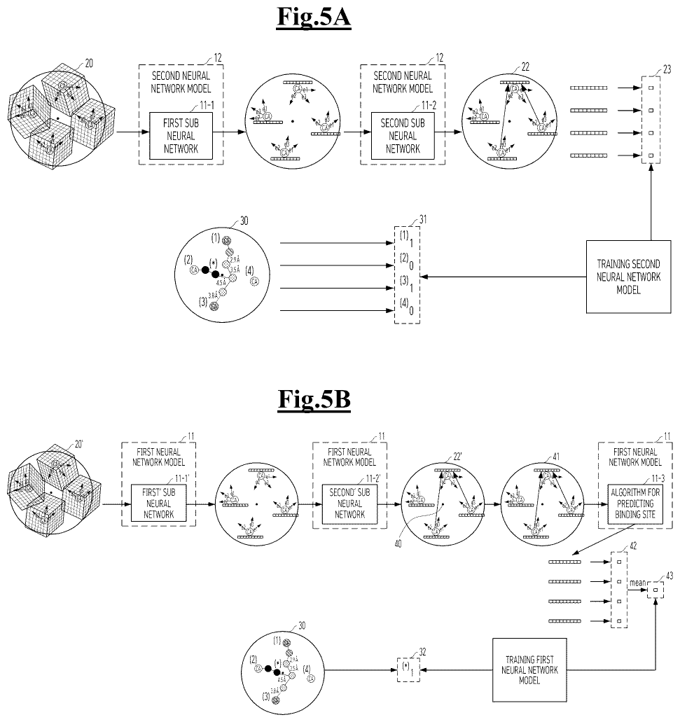

Meanwhile, the first neural network model may correspond to a model trained by an operation of obtaining first training data and first ground truth data corresponding to the first training data, an operation of predicting a training binding site based on the first training data using the first neural network model, and an operation of training the first neural network model based on the predicted training binding site and the first ground truth data. Specifically, the first neural network model may correspond to a model trained based on an operation in which the first neural network model shares some parameters with the trained second neural network model to be described below, and setting some parameters shared with the trained second neural network model under an initial condition, and an operation of performing training for the first neural network model based on the set initial condition. For example, the first neural network model may be trained through transfer learning after a training process of the second neural network model is performed, and hereinafter, a detailed description thereof will be described below through FIG. 5B.

According to an exemplary embodiment of the present disclosure, the computing device 100 uses the second neural network model for identifying the binding residue to predict the binding residue based on the candidate data filtered through step S120 (S130). In this case, the second neural network model for identifying the binding residue may mean a model that performs an operation of identifying binding residues which may be bound with a binding site center. Meanwhile, the second neural network model may share some parameters with the first neural network model. In this case, two neural network models sharing some parameters may mean that two models use several weights or parameters similarly, and the shared parameter may be used for transfer learning that trains one neural network model, and then uses some trained weights of the model for other related tasks, and may be used for hybrid models which combine and use two or more other model architectures, but the present disclosure is not limited thereto. Through this, the computing device 100 may reduce a size of the model and more effectively use the training data, and enhance efficiency of the model and reduce overfitting. Meanwhile, the second neural network model may include at least one of a first sub neural network for extracting a local feature of the binding site and a second sub neural network for globally aggregating the local features, which is included in the first neural network model. Specifically, the second neural network model may share at least one of the first sub neural network or the second sub neural network with the first neural network model. For example, the first sub neural network for extracting the local feature of the binding site may include a 3D convolutional neural network (CNN), and the second sub neural network for globally aggregating the local features may include a geometric attention layer, and the second sub neural network may be constituted by a plurality of geometrical self-attention layers.

The computing device 100 may input the protein and a filtered binding site center Ci′(i=1, . . . , n) into the second neural network model, and obtain a predicted binding residue Ri (i=1, . . . , n) corresponding to the filtered binding site center Ci′ (i=1, . . . , n) as the output. In this process, the computing device 100 may perform an operation of applying a grid alignment through the first sub neural network, and transform a surrounding of Ci′ (i=1, . . . , n) into an amino acid unit 3D grid set, and process the grids through the second neural network model and generate an output. In this case, even though rotation is applied to an input through a process of aligning the grids, SE-(3) immutability may be satisfied so that the output is not changed, and the SE-(3) immutability may mean conversion immutability in this space after a Euclidean space is extended to an SE(3) space which is an extended space. In this regard, since information such as the binding site of the protein structure is not changed and should be maintained regardless of a reference frame, additional processes such as using an axis aligned according to a orientation of the residue without using an arbitrary xyz axis when configuring the grid may be performed so that the SE-(3) immutability is satisfied in the exemplary embodiments of the present disclosure. Meanwhile, the 3D grid may mean that the space is divided into the grid, and each grid cell may have a predetermined size and a predetermined shape, and represent a position in a 3D space, and the computing device 100 may express a specific local environment of the protein by using the amino acid unit 3D grid set with respect to one or more candidate data. Additionally, the computing device 100 may perform an operation of randomly transforming the orientation of the residue through the second sub neural network in the training process of at least one of the first neural network or the second neural network. In this regard, the computing device 100 may increase diversity of geometrical information without fully forgetting an original residue orientation through random conversion for the training data in a training process to be described below, and accordingly obtain an effect of augmenting the training data. Meanwhile, the computing device 100 predicts the binding site and predicts the binding residue for the predicted binding site by using the first neural network model for detecting a binding site and the second neural network model for identifying a binding residue, which share some parameters to reduce the size of the model and more effectively use the training data, thereby more accurately predicting a binding pocket (binding residue and binding site) of the protein.

According to an exemplary embodiment of the present disclosure, the second neural network model may correspond to a model trained based on an operation of obtaining second training data and second ground truth data corresponding to the second training data, an operation of predicting a training binding residue based on the one or more training data using the second neural network model, and an operation of training the second neural network model based on the predicted training binding residue and the second ground truth data. Further, the training process of the second neural network model may be performed earlier than the training process for the first neural network model, and the trained second neural network model may be trained based on an operation of sharing some parameters with the first neural network model, and setting some parameters shared with the trained second neural network model under an initial condition by the first neural network model, and an operation of performing training for the first neural network model based on the set initial condition. For example, transfer learning may be performed in which the shared parameter included in the trained second neural network model is set as the initial condition of the process of training the first neural network model, and hereinafter, a detailed description thereof will be described below through FIGS. 5A and 5B. Meanwhile, expressions such as first, second, etc., disclosed throughout this specification are just a meaning for distinguishing components, and the present disclosure is not limited thereto.

FIG. 4 is a schematic view illustrating a process for predicting a binding site of a protein according to an exemplary embodiment of the present disclosure.

Referring to FIG. 4, the computing device 100 may receive “protein P and n which is the number of binding sites” (1). In this case, “ligand (drug or other molecules) li (i=1, . . . , n)” (1) corresponding to n binding sites may be obtained in an already known structure. Further, the computing device 100 may obtain one or more candidate binding sites and a center 2 of each of the candidate binding sites in the protein P (1) using an algorithm 10 for predicting the binding site in a protein structure. Specifically, the computing device 100 may find heavy atoms S1, . . . , Sm corresponding to an area which is likely to geometrically become the binding pocket (binding residue and binding site) on the surface of the protein P through the algorithm 10, and obtain C1, . . . , Cm 2 which are centers of the respective candidate binding sites by taking a center of an atomic mass for each of S1 to Sm. In this case, the computing device 100 approximates a local curvature to a radius of an alpha sphere which is a “sphere having four heavy atoms, but having no heavy atom therein” by using the algorithm 10 to obtain “one or more candidate binding sites and the center of each of the candidate binding sites” (2) of the protein P (1), and as an example of the algorithm 10, an algorithm for analyzing biological information related to the protein structure, such as Fpocket, and detecting a pocket (binding residue and binding site) of the protein may be used, but the present disclosure is not limited thereto, and various examples may be used. Through this, the computing device 100 may obtain the one or more candidate data (for example, C1, . . . , Cm) 2 including “one or more candidate binding sites and the center of each of the candidate binding sites” of the protein P (1) by using the algorithm 10.

The computing device 100 may filter the one or more candidate data (for example C1, . . . , Cm) 2 and obtain the filtered candidate data 3 by using a first neural network model 11 for detecting the binding site. In this case, the first neural network model 11 for detecting the binding site may mean a model which performs an operation of identifying the binding site which is a position bound with ligand (drug or other molecules) in a given protein. For example, the computing device 100 selects n candidate data among m candidate data (for example, C1, . . . , Cm) 2 by using the first neural network model 11 based on ligand (drug or other molecules) li (i=1, . . . , n)” 1 corresponding to n binding sites, respectively to obtain n filtered candidate data. In this case, the first neural network model may share some parameters with a second neural network model 12 to be described below. In this case, two neural network models sharing some parameters may mean that two models use several weights or parameters similarly, and the shared parameter may be used for transfer learning that trains one neural network model, and then uses some trained weights of the model for other related tasks, and may be used for hybrid models which combine and use two or more other model architectures, but the present disclosure is not limited thereto. Through this, the computing device 100 may reduce a size of the model and more effectively use the training data, and enhance efficiency of the model and reduce overfitting.

The first neural network model 11 may include a first sub neural network for extracting a local feature of the binding site and a second sub neural network for globally aggregating the local features. For example, the first sub neural network for extracting the local feature of the binding site may include a 3D convolutional neural network (CNN), and the second sub neural network for globally aggregating the local features may include a geometric attention layer. For example, the second sub neural network may be constituted by a plurality of geometric self-attention layers. Further, the first neural network model 11 may further include a third sub neural network for mapping the feature aggregated through the second sub neural network to a single scalar quantity, and the third sub neural network may be configured through a point-wise feed-forward layer and a mean-reduction operation, but the present disclosure is not limited to the example, and various exemplary embodiments may be used.

Specifically, the computing device 100 may calculate a druggability score for each of m candidate data (for example C1, . . . , Cm) 2 by using the first neural network model 11 for detecting the binding site, and obtain the n filtered candidate data (C1′, . . . , Cn′) 3 based on the druggability score for each of the candidate data (C1, . . . , Cm) 2. In this case, the druggability score may mean an index indicating a possibility that a drug will be bound to a specific site of the protein, and in the case of a pocket (binding residue and binding site) having a larger binding site and capable of effectively accommodating the drug, when a possibility that a specific chemical reaction will be caused or the drug will be bound to a specific atom or binding is high when the drug is bound, the druggability score may be calculated to be high in a case where the binding site is stably maintained in the protein. For example, the computing device 100 may calculate the druggability score in specific Ci (i=1, . . . , m) by using the protein and the candidate binding site center C1, . . . , Cm 2 as an input of the first neural network model 11, and filters n candidate binding site centers among the candidate binding site centers C1, . . . , Cm in the order of a higher calculated druggability score to obtain C1′, . . . , Cn′ 3 which are filtered candidate binding site centers. In this process, the computing device 100 may perform an operation of applying a grid alignment through the first sub neural network. Further, the computing device 100
1) transforms the surrounding of the candidate binding site center Ci (i=1, . . . , m) into an amino acid unit 3D grid set, and
2) uses the first sub neural network (for example, 3D CNN) to encode a local environment of residues of the surrounding of the candidate binding site center Ci (i=1, . . . , m).

In this regard, the 3D grid may mean that the space is divided into the grid, and each grid cell may have a predetermined size and a predetermined shape, and represent a position in a 3D space, and the computing device 100 may express a specific local environment of the protein by using the amino acid unit 3D grid set with respect to one or more candidate data. Further, each grid of the surrounding of the candidate binding site center Ci (i=1, . . . , m) may mean residues sufficiently close to Ci, and for example, may mean residues positioned at a distance within 17 angstrom of the surrounding of Ci, but is not limited thereto.

Thereafter, the computing device 100 processes the encoded grids through the second sub neural network and the third sub neural network included in the first neural network model 11 to generate C1′, . . . , Cn′ 3 which is the filtered candidate binding site center as the output. Specifically, the computing device 100 may globally aggregate the “local features of the grids encoded through 3D CNN” by using the second sub neural network including a plurality of geometric self-attention layers. For example, xi (i=1, . . . , m) may mean the “local features of the grids encoded through the 3D CNN” or a hidden vector sequence of a previous attention layer, and in {(Ri,ti)} (i=1, . . . , m), Ri may mean a orientation of the residue and ti may mean a center of the residue, and the computing device 100 transforms xi (i=1, . . . , m) into x′i (i=1, . . . , m) which is another hidden vector sequence through the geometric self-attention layer. A process of globally aggregating xi (i=1, . . . , m) through the plurality of geometric self-attention layers by the computing device 100 may be performed as follows.

1) First, the computing device 100 may obtain a query, key, and value vectors required for calculation as follows.

1-1) Standard query vector qh(i) and geometric query vector qhp(i)

1-2) Standard key vector kh(i) and geometric key vector khp(i)

1-3) Standard value vector vh(i) and geometric value vector vhp(i) In this case, h may mean “head”, and p may mean “point of attention”.

2) Further, the computing device 100 may obtain an attention weight through the following process.

2-1) The attention weight Wh(ij) may be obtained based on a standard item Wstandard(ij) and a geometric item Wgeometric(ij).

2-2) The standard item Wstandard(ij) may be obtained based on an inner product between the standard query vector qh(i) and the standard key vector kh(i).

Specifically, an attention weight from an i-th token to a j-th token may be calculated by a linear combination of the standard items, and calculated as in the following calculation equation.

$${w}_{ij}^{h , standard} = \frac{1}{\sqrt{{d}_{hidden}}} {q}_{i}^{h} \cdot {k}_{j}^{h}$$

2-3) The geometric item Wgeometric(ij) may be obtained based on a distance between global coordinates of the geometric query vector qhp(i) and the geometric key vector khp(i).

Specifically, geometric item Wgeometric(ij) may be calculated as in the following equation. In this case, Ti may mean reducing and expressing {Ri,ti}, and mean a local frame related to the residue.

$${w}_{ij}^{h , geometric} = \frac{1}{\sqrt{{N}_{points}}} {\sum }_{p} ‖ {T}_{i} {q}_{i}^{hp} \cdot {T}_{j} {k}_{j}^{hp} ‖$$

2-4) Therefore, the attention weight Wh(ij) considering the standard item and the geometric item may be calculated as follows.

$${w}_{ij}^{h} = {softmax}_{j} ( \frac{1}{\sqrt{2}} ( {w}_{ij}^{h , standard} - log ( 1 + {γ}^{h} ) {w}_{ij}^{h , geometric} ) )$$

3-1) The computing device 100 may obtain a standard value vector oh(i) aggregated based on the standard item Wstandard(ij) and the standard value vector vhp(i) of the attention weight, and the standard value vector oh(i) may be expressed as in the following equation.

$${o}_{i}^{h} = {\sum }_{j} {w}_{ij}^{h} {v}_{j}^{h}$$

3-2) The computing device 100 may obtain a global coordinate ohp(i) aggregated based on the geometric item Wgeometric(ij) and the geometric value vector vh(i) of the attention weight, and the global coordinate ohp(i) may be expressed as in the following equation.

$${o}_{i}^{hp} = {T}_{i}^{- 1} ( {\sum }_{j} {w}_{ij}^{h} {T}_{j} {v}_{j}^{hp} )$$

3-3) The computing device 100 may obtain x′i which is an updated hidden vector sequence calculated based on the aggregated standard value vector oh(i) and the aggregated global coordinate ohp(i), and the updated hidden vector sequence x′i may be expressed as in the following equation.

$${x}_{i}^{′} = {f}_{final} ( {concat}_{h , p} ( {o}_{i}^{h} , {o}_{i}^{hp} , ‖ {o}_{i}^{hp} ‖ ) )$$

Through this, the computing device 100 may update the hidden vector through the plurality of geometric self-attention layers included in the second sub neural network, calculate the attention weight by considering standard information and geometric information jointly, and generate the updated hidden vector to excellently reflect information on given input data. Meanwhile, the computing device 100 may perform an operation of randomly transforming the orientation of the residue through the second sub neural network in the training process of at least one of the first neural network or the second neural network. Through this, the computing device 100 may increase diversity of geometrical information without fully forgetting an original residue orientation through random conversion for the training data in a training process to be described below, and accordingly obtain an effect of augmenting the training data.

Thereafter, the computing device 100 may obtain the n filtered candidate data (C1′, . . . , Cn′) 3 by using a third sub neural network for mapping the feature aggregated through the second sub neural network to a single scalar quantity. In this case, the third sub neural network may be configured through a point-wise feed-forward layer and a mean-reduction operation, but the present disclosure is not limited thereto.

According to an exemplary embodiment of the present disclosure, the computing device 100 uses the second neural network model 12 for identifying the binding residue to predict the binding residue 4 based on the n filtered candidate data (C1′, . . . , Cn′) 3. In this case, the second neural network model 12 for identifying the binding residue to predict the binding residue may mean a model that performs an operation of identifying binding residues which may be bound with a binding site center. Meanwhile, the second neural network model 12 may share some parameters with the first neural network model 11. In this case, two neural network models sharing some parameters may mean that two models use several weights or parameters similarly, and the shared parameter may be used for transfer learning that trains one neural network model, and then uses some trained weights of the model for other related tasks, and may be used for hybrid models which combine and use two or more other model architectures, but the present disclosure is not limited thereto. Through this, the computing device 100 may reduce a size of the model and more effectively use the training data, and enhance efficiency of the model and reduce overfitting. Meanwhile, the second neural network model 12 may include at least one of a first sub neural network for extracting a local feature of the binding site and a second sub neural network for globally aggregating the local features, which is included in the first neural network model 11. Specifically, the second neural network model 12 may share at least one of the first sub neural network or the second sub neural network with the first neural network model, and for example, the second neural network model 12 may share the 3D convolutional neural network (CNN) included in the first sub neural network for extracting the local feature of the binding site with the first neural network model 11, and share some of the plurality of geometric self-attention layers included in the second sub neural network for globally aggregating the local features. However, in addition to the example, various exemplary embodiments may be used in which the second neural network model 12 shares at least one of the first sub neural network or the second sub neural network with the first neural network model. The computing device 100 may input a filtered binding site center Ci′(i=1, n) 3 into the second neural network model 12, and obtain a predicted binding residue Ri (i=1, . . . , n) 4 corresponding to the filtered binding site center Ci′ (i=1, . . . , n) as the output. In this process, the computing device 100 may perform an operation of applying a grid alignment through the first sub neural network, and transform a Ci′ (i=1, . . . , n) surrounding into an amino acid unit 3D grid set, and process the grids through the second neural network model 12 and generate an output. In this case, even though rotation is applied to an input through a process of aligning the grids, SE-(3) immutability may be satisfied so that the output is not changed, and the SE-(3) immutability may mean conversion immutability in this space after a Euclidean space is extended to an SE(3) space which is an extended space. In this regard, since information such as the binding site of the protein structure is not changed and should be maintained regardless of a reference frame, additional processes such as using an axis aligned according to a orientation of the residue without using an arbitrary xyz axis when configuring the grid may be performed so that the SE-(3) immutability is satisfied in the exemplary embodiments of the present disclosure. Meanwhile, the 3D grid may mean that the space is divided into the grid, and each grid cell may have a predetermined size and a predetermined shape, and represent a position in a 3D space, and the computing device 100 may express a specific local environment of the protein by using the amino acid unit 3D grid set with respect to one or more candidate data. Additionally, the computing device 100 may perform an operation of randomly transforming the orientation of the residue through the second sub neural network in the training process of at least one of the first neural network or the second neural network. In this regard, the computing device 100 may increase diversity of geometrical information without fully forgetting an original residue orientation through random conversion for the training data in a training process to be described below, and accordingly obtain an effect of augmenting the training data.

Specifically, the computing device 100 uses some parameters shared with the first neural network model 11 in the second neural network model 12 to obtain x″i (i=1, . . . , n) which is a updated hidden vector sequence for the filtered binding site center Ci′ (i=1, . . . , n), and process the x″i (i=1, . . . , n) through the point-wise feed-forward layer without the mean-reduction operation, thereby determining whether the residue is a binding site residue. In this case, the process of obtaining x″i (i=1, . . . , n) which is the updated hidden vector sequence may be described above in the present disclosure, or various exemplary embodiments to be described below may be used. Therefore, the computing device 100 may obtain a predicted binding residue Ri (i=1, . . . , n) 4 corresponding to the filtered binding site center Ci′ (i=1, . . . , n) as the output of the second neural network model 12. Through this, the computing device 100 predicts the binding site and predicts the binding residue for the predicted binding site by using the first neural network model 11 for detecting a binding site for predicting a binding site and the second neural network model 12 for identifying a binding residue, which share sharing some parameters to reduce the size of the model and more effectively use the training data, thereby more accurately predicting a binding pocket (binding residue and binding site) of the protein. Meanwhile, the first neural network model 11 and the second neural network model 12 may be trained through transfer learning, and hereinafter, a detailed description thereof will be described below through FIGS. 5A and 5B. Meanwhile, expressions such as first, second, etc., disclosed throughout this specification are just a meaning for distinguishing components, and the present disclosure is not limited thereto.

FIG. 5A is a schematic view for describing a process of training a second neural network model for identifying a binding residue according to an exemplary embodiment of the present disclosure.

Referring to FIG. 5A, the second neural network model 12 may correspond to a model trained based on an operation of obtaining second training data 20 and second ground truth data 31 corresponding to the second training data 20, an operation of predicting a training binding residue 23 based on one or more training data by using the second neural network model 12, and an operation of training the second neural network model 12 based on the predicted training binding residue 23 and the second ground truth data 31. In this case, the second training data 20 may include a candidate binding site of a training protein and a center 20 thereof, and the second ground truth data 31 may include a ground truth binding residue corresponding to the second training data 20 in the reference protein 30.

For example, the computing device 100 may transform a surrounding of the candidate binding site center 20 of the training protein into the amino acid unit 3D grid set, and encode a local environment of residues of the surrounding of the “candidate binding site center 20 of the training protein” by using a first sub neural network (for example, 3D CNN) 11-1. In this regard, the 3D grid may mean that the space is divided into the grid, and each grid cell may have a predetermined size and a predetermined shape, and represent a position in a 3D space, and the computing device 100 may express a specific local environment of the protein by using the amino acid unit 3D grid set with respect to the second training data 20. Further, the grid of the surrounding of the “candidate binding site center 20 of the training protein” may mean residues sufficiently close to the “the candidate binding site center 20 of the training protein”, and for example, may mean residues positioned at a distance within 17 angstrom of the surrounding of the “candidate binding site center 20 of the training protein”, but is not limited thereto.

Thereafter, the computing device 100 processes the encoded grid 21 through a second sub neural network 11-2 included in the second neural network model 12, and the point-wise feed forward layer to predict the training binding residue 23. In this case, the point-wise feed forward layer may mean a layer that individually processes an input for each position in the sequence, and to this end, a 1×1 convolutional layer and an activation function (e.g., ReLU) may be used, but the present disclosure is not limited thereto. Specifically, the computing device 100 may globally aggregate the “local features of the grids 21 encoded through 3D CNN” by using the second sub neural network 11-2 including the plurality of geometric self-attention layers, and predict the training binding residue 23 by processing the aggregated features 22 through the point-wise feed forward layer. In this case, the computing device 100 may perform an operation of randomly transforming the orientation of the residue in the training process of at least one of the first neural network or the second neural network through the second sub neural network 11-2. Through this, the computing device 100 randomly transforms the second training data 20 in the training process to increase diversity of geometrical information without fully forgetting an original residue orientation, and accordingly obtain an effect of augmenting the second training data 20 in the training process of the second neural network model. Specifically, according to an exemplary embodiment of the present disclosure, an existing augmentation method such as rotation when the computing device 100 applies the SE-(3) immutability through the grid alignment in the binding residue prediction process may not influence a training result. Therefore, the computing device 100 randomly transforms the orientation of the residue of the second training data 20 in the training process of at least one of the first neural network or the second neural network to increase diversity of geometric information of the training data in spite of the SE-(3) immutability.

The computing device 100 may train the second neural network model based on the predicted training binding residue 23 and the second ground truth data 31. For example, in respect to the second ground truth data 31, when “residue #1 and residue #3 are the binding residues”, if the predicted training binding residue 23 is not “residue #1 and residue #3”, a loss function may be calculated based thereon, and accordingly, the second neural network model 12, and the first and second sub neural network models 11-1 and 11-2 included therein may be trained. Meanwhile, the second neural network model 12 may share some parameters with the first neural network model 11. In this case, two neural network models sharing some parameters may mean that two models use several weights or parameters similarly, and the shared parameter may be used for transfer learning that trains one neural network model, and then uses some trained weights of the model for other related tasks, but the present disclosure is not limited thereto. For example, the training process of the first neural network model 11 may be performed through the transfer learning after the training process of the second neural network model 12 is performed, and hereinafter, a detailed description thereof will be described below through FIG. 5B.

FIG. 5B is a schematic view for describing a process of training a first neural network model for detecting a binding site, which shares some parameters with a second neural network model according to an exemplary embodiment of the present disclosure. Referring to FIG. 5B, the computing device 100 may train the first neural network model 11 for detecting the binding site after performing the training process of the second neural network model 12 described above through FIG. 5A above.

Specifically, the first neural network model 11 may share some parameters with the trained second neural network model 12, the computing device 100 may set some parameters shared with the trained second neural network model 12′ of the first neural network model 11 as an initial condition of training, and train the first neural network model 11 based on the set initial condition. For example, the first neural network model 11 may include at least one of a trained first′ sub neural network 11-1′ or a trained second′ sub neural network 11-2′ included in the trained second neural network model 12′.

In this regard, the computing device 100 may correspond to a model trained based on an operation of obtaining first training data 20′ and first ground truth data 32 corresponding to the first training data 20′ by using the first neural network model 11 which shares some parameters with the trained second neural network model 12′, an operation of predicting the training binding site 43 based on the first training data 20′ by using the first neural network model 11, and an operation of training the second neural network model 12 based on the predicted training binding site 43 and the first ground truth data 32. In this case, the first training data 20 may include a candidate binding site of a training protein and centers 20′ thereof, and the first ground truth data 32 may include a ground truth binding site center 32 corresponding to the first training data 20′ in the reference protein 30.

For example, the computing device 100 may transform a surrounding of the candidate biding site centers 20′ of the training protein into the amino acid unit 3D grid set, and encode a local environment of the surrounding of the “candidate binding site center 20′ of the training protein” by using a trained first sub neural network (for example, 3D CNN) 11-1′. In this regard, the 3D grid may mean that the space is divided into the grid, and each grid cell may have a predetermined size and a predetermined shape, and represent a position in a 3D space, and the computing device 100 may express a specific local environment of the protein by using the amino acid unit 3D grid set with respect to the first training data 20′. Further, the grid of the surrounding of the “candidate binding site center 20′ of the training protein” may mean environments of the surrounding sufficiently close to the “the candidate binding site center 20′ of the training protein”, and for example, may mean an environment at a distance within 17 angstrom of the surrounding of the “candidate binding site center 20′ of the training protein”, but is not limited thereto.

Thereafter, the computing device 100 processes the encoded grid 21′ through a second′ sub neural network 11-2′ and a third sub neural network 11-3 trained, which are included in the first neural network model 11 to predict the training binding site 43. In this case, the third sub neural network 11-3 may include the point-wise feed forward layer which individually processes an input for each position in the sequence, and to this end, a 1×1 convolutional layer and an activation function (e.g., ReLU) may be used, but the present disclosure is not limited thereto. Additionally, the computing device 100 performs a mean-reduction operation of calculating a mean value in a data set given through the third sub neural network 11-3 to predict the training binding site 43. Specifically, the computing device 100 may globally aggregate the “local features of the grids 21′ encoded through 3D CNN” by using the trained second sub neural network 11-2′ including “the plurality of geometric self-attention layers shared with the trained second neural network model 12′”, and process the aggregated features 22′ and obtain the additionally aggregated features 41 by using “an additional geometric self-attention layer not shared with the trained second neural network model 12”. In this case, the computing device 100 may perform embedding of a reference point 40 in order to maintain and track identification and position information of a point when inputting the aggregated features 22′ into “the additional geometric self-attention layer not shared with the trained second neural network model 12′”, and additionally input the embedding of the reference point 40. Further, the computing device 100 processes the additionally aggregated features 41 through the point-wise feed forward layer and the mean-reduction operation using the third sub neural network 11-3 to predict the training binding site 43. In this case, the computing device 100 may perform an operation of randomly transforming the orientation of the residue through the trained second′ sub neural network 11-2′ in the training process of at least one of the first neural network or the second neural network. Through this, the computing device 100 randomly transforms the first training data 20′ in the training process to increase diversity of geometrical information without fully forgetting an original residue orientation, and accordingly obtain an effect of augmenting the first training data 20′ in the training process of the first neural network model 11. Specifically, according to an exemplary embodiment of the present disclosure, an existing augmentation method such as rotation when the computing device 100 applies the SE-(3) immutability through the grid alignment in the binding residue prediction process may not influence a training result. Therefore, the orientation of the residue is randomly transformed in the first training data 20′ to increase the diversity of the geometric information of the training data in spite of the SE-(3) immutability.

The computing device 100 may train the first neural network model 11 based on the predicted training binding site 43 and the first ground truth data 32. For example, when the first ground truth data 32 is “a center of binding site #1”, if the predicted training binding site 43 is not “the center of binding site #1”, the loss function may be calculated based thereon, and accordingly, the first neural network model 11, and the first and second sub neural network models 11-1′ and 11-2′ included therein may be additionally trained, and the third sub neural network model 11-3, and a parameter which is not shared with the second neural network model may be trained.

In this regard, the transfer learning performed through an exemplary embodiment of the present disclosure may obtain the following technical effect.

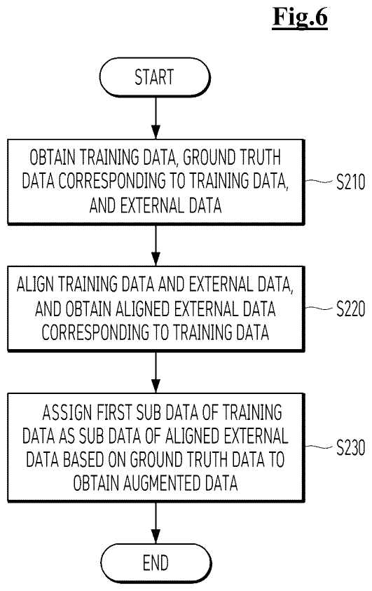

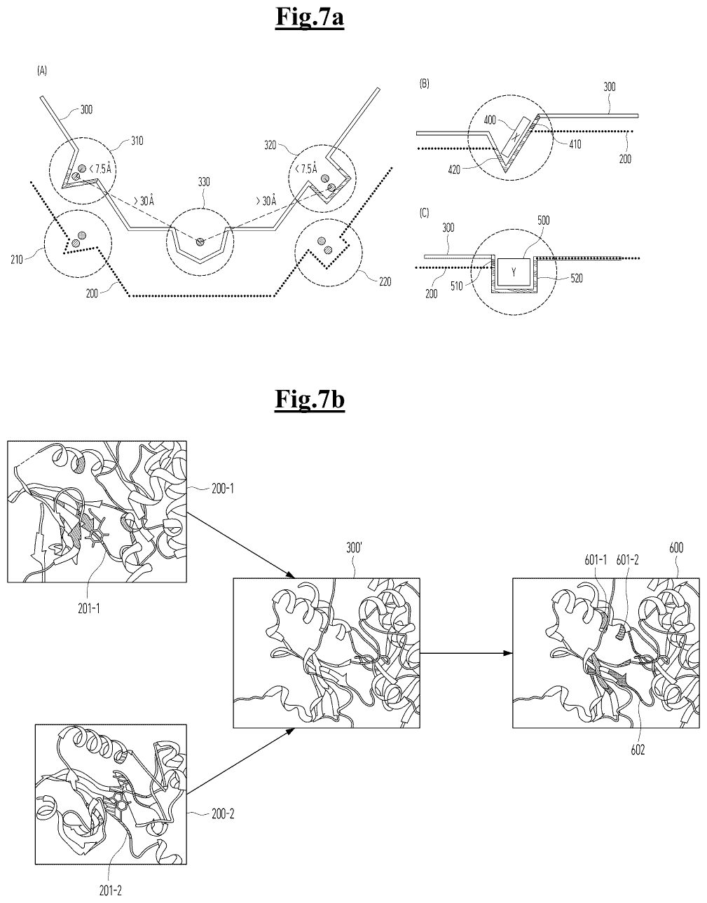

First, the first ground truth data 32 for the first neural network model 11 for detecting the binding site has only one ground truth label per one binding site, but the second ground truth data 31 for the second neural network model 12 for identifying the binding residue may have several ground truth labels per one binding site. Therefore, the computing device 100 first train the second neural network model 12, and then perform the training process of the first neural network model 11 by using the shared parameter as the initial condition to increase training efficiency by using the ground truth data for the second neural network model 12 which is more sufficient than the first neural network model 11 for detecting the binding site. Further, since the binding site of the protein may be determined based on a pattern of the binding residue, some parameters of the trained second neural network model 12′ which may excellently identify the pattern of the binding residue are used as the initial condition in the training process of the first neural network model to increase a prediction possibility of the binding site. Meanwhile, according to an exemplary embodiment of the present disclosure, as the first and second training data, and the first and second ground truth data corresponding to the first and second training data, respectively, augmented data may be used according to another exemplary embodiment of the present disclosure, and hereinafter, a specific process of obtaining the augmented data will be described below through FIGS. 6 to 7B.

FIG. 6 is a flowchart illustrating a method for obtaining augmented data for training at least one of a first neural network model for detecting a binding site or a second neural network model for identifying a binding residue according to an exemplary embodiment of the present disclosure.

According to an exemplary embodiment of the present disclosure, the computing device 100 may obtain training data, ground truth data corresponding to the training data, and external data (S210). In this case, the training data may include a training protein structure, and include one or more candidate binding sites of the protein and centers of the respective candidate binding sites, and the ground truth data corresponding to the training data may include at least one of a ground truth binding site or a ground truth binding residue. In this case, the ground truth binding site may include data in which is a label is designated as a positive number and a negative number for the center of the binding site of the protein, and the ground truth binding residue may include data indicating a possibility that a surrounding protein residue will be a ligand binding residue for the binding site in which the positive number label is designated, but the present disclosure is not limited thereto. Further, the external data may mean a protein obtained in an external database in which a ground truth label of the binding site or the binding residue is not designated, but is not limited thereto. For example, the training data and the ground truth data may be obtained in scPDB which is a seed database of a protein-ligand complex, and the external data may be obtained in a database of a single chain structure. Meanwhile, the training data, the ground truth data, and the external data may be used in the process of obtaining the augmented data, and hereinafter, a detailed description will be described below through FIGS. 7A and 7B.

According to an exemplary embodiment of the present disclosure, the computing device 100 may align the training data and the external data obtained through step S210, and obtain aligned external data corresponding to the training data (S220). In this case, a process of aligning the training data and the external data may include a protein sequence aligning process, and the protein sequence aligning process may mean a process of aligning the protein using a sequence alignment algorithm in order to compare and analyze a similarity between various protein sequences, but is not limited thereto. Specifically, the computing device 100 may obtain an amino acid sequence associated with the training data, and when the obtained amino acid sequence is conserved in the aligned external data with the training data, the computing device 100 may obtain the aligned external data corresponding to the training data. Specifically, the computing device 100 may obtain a training protein chain as the training data, and obtain one or more amino acid sequences associated therewith, and obtain an external protein chain as the external data. Further, the computing device 100 may configure the training protein chain by query chain x, and configure the external protein chain by target chain y, and when amino acid sequence l associated with the query chain x is conserved in aligned target chain y, the computing device 100 may obtain the aligned target chain y as the aligned external data. Specifically, when the amino acid sequence l associated with the query chain x is conserved at 50% or more in the aligned target chain y, the aligned target chain y may be obtained as the aligned external data, but various exemplary embodiments may be used in addition to the example of 50%. Meanwhile, the aligned external data with the training data may be used in the process of obtaining the augmented data, and hereinafter, a detailed description will be described below through FIGS. 7A and 7B.

According to an exemplary embodiment of the present disclosure, the computing device 100 assign first sub data of the training data as sub data of the aligned external data through step S220 based on the ground truth data obtained through step S210 to obtain the augmented data (S230). In this case, the augmented data may be used in the process of training at least one of the first neural network model for detecting the binding site or the second neural network model for identifying the binding residue. Further, assigning the first sub data of the training data as the sub data of the aligned external data may mean labeling the first sub data to some (corresponding to some of the training data) of the aligned external data based on a ground truth label for some of the training data.

For example, the computing device 100 assign the center of the ground truth binding site to the center of the binding site of the aligned external data to obtain first augmented data. Specifically, the computing device 100 uses an algorithm for predicting the binding site in the protein structure for the external data to obtain the center of the candidate binding site, obtain the center of the binding site of the aligned external data corresponding to the training data, and measure a distance between the obtained center of the candidate binding site of the aligned external data and the center of the binding site corresponding to the training data. In this case, as an example of the algorithm for predicting the binding site in the protein structure, an algorithm for analyzing biological information related to the protein structure, such as Fpocket, and detecting a pocket (binding residue and binding site) of the protein may be used, but the present disclosure is not limited thereto, and various examples may be used.

When the measured distance is within a predetermined threshold, the center of the ground truth binding site is assigned to the center of the binding site of the aligned external data corresponding to the training data to obtain the first augmented data. Alternatively, the computing device 100 may calculate a ratio at which the binding residue of the aligned external data corresponding to the aligned training data corresponds to the ground truth binding residue, and when the calculated ratio is equal to or more than a predetermined threshold, the computing device 100 assign the center of the ground truth binding site to the center of the binding site of the aligned external data corresponding to the training data to obtain the first augmented data. In this regard, the first augmented data may be used in the process of training the first neural network model for detecting the binding site according to an exemplary embodiment of the present disclosure, and hereinafter, a specific process of obtaining the first augmented data will be described below through FIG. 7A.

As yet another example, the computing device 100 assigns “a first ground truth binding residue for a first training protein structure” included in the ground truth data to the binding residue of the aligned external data corresponding to the training data to obtain second augmented data. Alternatively, the computing device 100 assigns “the first ground truth binding residue for the first training protein structure” included in the ground truth data to a first binding residue of the aligned external data corresponding to the training data to obtain a first assigned binding residue, and assigns “a second ground truth binding residue for a second training protein structure” included in the ground truth data to a second binding residue of the aligned external data corresponding to the training data to obtain a second assigned binding residue, and may obtain the second augmented data based on the first assigned binding residue and the second assigned binding residue. Meanwhile, there is a technical effect in which the computing device 100 augments the training data based on protein homology through an exemplary embodiment of the present disclosure to increase the diversity of the training data, thereby preventing overfitting. In this regard, the second augmented data may be used in the process of training the second neural network model for identifying the binding residue according to an exemplary embodiment of the present disclosure, and hereinafter, a specific process of obtaining the second augmented data will be described below through FIG. 7B. Meanwhile, expressions such as first, second, etc., disclosed throughout this specification are just a meaning for distinguishing components, and the present disclosure is not limited thereto.

FIG. 7A is a schematic view for describing a process of obtaining first according to an exemplary embodiment of the present disclosure.

Referring to FIG. 7A, the computing device 100 may align a training protein structure 200 and an external protein structure, and obtain an aligned external protein structure 300 corresponding to the training protein structure 200. In this case, a process of aligning the training protein structure 200 and the external protein structure may include a protein sequence aligning process, and the protein sequence aligning process may mean a process of aligning the protein using a sequence alignment algorithm in order to compare and analyze a similarity between various protein sequences, but is not limited thereto.

Specifically, the computing device 100 may obtain amino acid sequences 400 and 500 associated with the training data, and when the obtained amino acid sequences 400 and 500 are conserved in the training protein structure and the aligned external protein structure, the computing device 100 may obtain an aligned external protein structure 300 corresponding to the training protein structure 200. Specifically, the computing device 100 may obtain a training protein chain 200 as the training data, and obtain one or more amino acid sequences 400 and 500 associated therewith, and obtain an external protein chain as the external data. Further, the computing device 100 may configure the training protein chain 200 by query chain x, and configure the external protein chain by target chain y, and when amino acid sequence 1 400 or 500 associated with the query chain x is conserved in aligned target chain y, the computing device 100 may obtain the aligned target chain y as the aligned external protein structure 300.

Specifically, first, referring to (B) of FIG. 7A, a degree at which amino acid sequence la 400 associated with the training protein chain 200 is conserved in the target chain y may be represented as a bright area 420 and a degree at which the amino acid sequence la 400 is not conserved may be represented as a dark area 410. Accordingly, the computing device 100 since the amino acid sequence la 400 associated with the query chain x is conserved at 50% or more in the aligned target chain y (that is, since the bright area 420 is equal to or larger than the dark area 410), the aligned target chain y may be obtained as the aligned external protein structure 300, but various exemplary embodiments may be used in addition to the example of 50%.

Similarly, referring to (C) of FIG. 7A, a degree at which amino acid sequence lb 500 associated with the training protein chain 200 is conserved in the target chain y may be represented as a bright area 520 and a degree at which the amino acid sequence lb 500 is not conserved may be represented as a dark area 510. Accordingly, since the amino acid sequence lb 500 associated with the query chain x is conserved at 50% or more in the aligned target chain y (that is, since the bright area 520 is equal to or larger than the dark area 510), the computing device 100 may obtain the aligned target chain y as the aligned external protein structure 300, but various exemplary embodiments may be used in addition to the example of 50%.

The computing device 100 assigns a center 210 or 220 of the ground truth binding site included in the ground truth data to a center 310, 320, or 330 of the binding site of the aligned external protein structure 300 to obtain first augmented data. Specifically, the computing device 100 uses an algorithm for predicting the binding site in the protein structure for the external protein structure 300 to obtain the center 310, 320, or 330 of the candidate binding site, obtain the center of the binding site of the aligned external protein structure corresponding to the training data, and measure a distance between the obtained center (a circle of 310, 320, or 330) of the candidate binding site of the aligned external protein structure and the center (a lower circle of 310 or 320) of the binding site corresponding to the training data. In this case, as an example of the algorithm for predicting the binding site in the protein structure, an algorithm for analyzing biological information related to the protein structure, such as Fpocket, and detecting a pocket (binding residue and binding site) of the protein may be used, but the present disclosure is not limited thereto, and various examples may be used. Further, when the measured distance is within a predetermined threshold, the computing device 100 assigns a label to the center 310, 320, or 330 of the binding site included in the aligned external protein structure 300 based on the center 210 or 220 of the ground truth binding site to obtain the first augmented data.

Referring to (A) of FIG. 7A, the computing device 100 may measure a distance between a center (a lower circle of 310) of a first corresponding binding site of the aligned external protein structure corresponding to the training data and a center (an upper circle of 310) of a first candidate binding site of the aligned external protein structure 300, and when the distance between the center (the lower circle of 310) of the first corresponding binding site and the center (the upper circle of 310) of the first candidate binding site is less than 7.5 angstrom, the computing device 100 may assign a positive number label to the center (the upper circle of 310) of the first candidate binding site. Similarly, the computing device 100 may measure a distance between a center (a lower circle of 320) of a second corresponding binding site of the aligned external protein structure corresponding to the training data and a center (an upper circle of 320) of a second candidate binding site included in the aligned external protein structure 300, and when the distance between the center (the lower circle of 320) of the second corresponding binding site and the center (the upper circle of 320) of the second candidate binding site is less than 7.5 angstrom, the computing device 100 may assign a positive number label to the center (the upper circle of 320) of the second candidate binding site.

On the contrary, the computing device 100 may measure the distances between the centers (the lower circles of 310 and 320) of the first and second corresponding binding sites and a center (a circle of 330) of a third candidate binding site included in the aligned external protein structure 300, and when the distances between the center (the lower circles of 310 and 320) of the first and second corresponding binding sites and the center (the circle of 330) of the third candidate binding site is equal to or more than 30 angstrom, the computing device 100 may assign a negative number label to the center (the circle of 330) of the third candidate binding site. Through this, the computing device 100 may obtain the first augmented data in which the label is assigned to the aligned external data 300, and the first augmented data may be used in the process of training the first neural network model for detecting the binding site according to an exemplary embodiment of the present disclosure, and there is a technical effect in which the first augmented data is augmented based on the protein homology to increase the diversity of the training data, thereby preventing the overfitting. Meanwhile, second augmented data to be described below may be used in the process of training the second neural network model for identifying the binding residue according to an exemplary embodiment of the present disclosure, and hereinafter, a specific process of obtaining the second augmented data will be described below through FIG. 7B.

FIG. 7B is a schematic view for describing a process of obtaining second augmented data for training a second neural network model for identifying a binding residue according to an exemplary embodiment of the present disclosure.

According to an exemplary embodiment of the present disclosure, the computing device 100 may align a training protein structure 200 and an external protein structure, and obtain an aligned external protein structure 300′ corresponding to the training protein structure 200. In this case, a process of aligning the training protein structure 200 and the external protein structure may include a protein sequence aligning process, and the protein sequence aligning process may mean a process of aligning the protein using a sequence alignment algorithm in order to compare and analyze a similarity between various protein sequences, but is not limited thereto.

Specifically, the computing device 100 may obtain an amino acid sequence associated with the training data, and when the obtained amino acid sequence is conserved in the training protein structure and the aligned external protein structure, the computing device 100 may obtain an aligned external protein structure 300′ corresponding to the training protein structure 200. Specifically, the computing device 100 may obtain a training protein chain 200 as the training data, and obtain one or more amino acid sequences associated therewith, and obtain an external protein chain as the external data. Further, the computing device 100 may configure the training protein chain 200 by query chain x, and configure the external protein chain by target chain y, and when amino acid sequence 1 associated with the query chain x is conserved in aligned target chain y, the computing device 100 may obtain the aligned target chain y as the aligned external protein structure 300′.

Referring to FIG. 7B, the computing device 100 assigns “a first ground truth binding residue 201-1 for a first training protein structure 200-1” included in the ground truth data to the binding residue corresponding to the training data included in the aligned external data 300′ to obtain second augmented data 600. That is, the computing device 100 assigns at least one of “a first ground truth binding residue 201-1 for a first training protein structure 200-1” or “a second ground truth binding residue 201-2 for a second training protein structure 200-2” included in the ground truth data to the binding residue corresponding to the training data included in the aligned external data 300′ to obtain the second augmented data 600.

According to another exemplary embodiment of the present disclosure, the computing device 100 assigns “the first ground truth binding residue 201-1 for the first training protein structure 200-1” included in the ground truth data to a first binding residue corresponding to the training data included in the aligned external data to obtain a first assigned binding residue 601-1, and assigns “the second ground truth binding residue 201-2 for the second training protein structure 200-2” included in the ground truth data to a second binding residue corresponding to the training data included in the aligned external data 300′ to obtain a second assigned binding residue 601-2, and may obtain the second augmented data 600 based on the first assigned binding residue 601-1 and the second assigned binding residue 601-2.

Specifically, the computing device 100 may assign a label of 0.5 to the binding residue corresponding to the first assigned binding residue 601-1 among the binding residues included in the aligned external data 300′, and also assigned the label of 0.5 to the second assigned binding residue 601-2. Further, the computing device 100 may assign a label of 1 to a redundant binding residue 602 which is a part where the first assigned binding residue 601-1 and the second assigned binding residue 601-2 are redundant. Through this, since there is a high probability that a part where there are a lot of redundant binding residues among several ground truth data will correspond to the binding residue, the second augmented data 600 may be obtained by considering this. However, the present disclosure is not limited to the examples of 0.5 and 1, and various examples may be used. Meanwhile, there is a technical effect in which the computing device 100 augments the training data based on protein homology through an exemplary embodiment of the present disclosure to increase the diversity of the training data, thereby preventing overfitting.

Meanwhile, an experimental result through the exemplary embodiments of the present disclosure is described through the following tables.

TABLE 1
<Table showing measurement of a mean of binding residues bound to the closest ligand>

| | scPDB(held-out) | COACH420 | HOLO4k | CHEN-holo | CHEN- [symbol] |
|---|---|---|---|---|---|
| DeepSurf | 0.288 ± 0.007 | 0.194 ± 0. [symbol] | 0.207 ± 0.003 | 0.104 ± 0.003 | 0.085 ± 0.005 |
| Ka [symbol] | 0.2 [symbol] ± 0.000 | 0.183 ± 0. [symbol] | 0.146 ± 0.000 | 0.101 ± 0.000 | 0.092 ± 0.000 |
| Deeppocket | 0.440 ± 0.002 | 0.313 ± 0.003 | 0.277 ± 0.03 | 0.190 ± 0.00 [symbol] | 0.186 ± 0.003 |
| Ours | 0.490 ± 0.003 | 0.398 ± 0.004 | 0.346 ± 0.002 | 0.287 ± 0.004 | 0.264 ± 0.004 |
| Ours(BERT) | 0.430 ± 0.008 | 0. [symbol] ± 0.010 | 0.294 ± 0.005 | 0.228 ± 0.007 | 0.206 ± 0.00 [symbol] |
| Ours(no CNN) | 0.4 [symbol] 7 ± 0.00 [symbol] | 0. [symbol] ± 0.005 | 0.315 ± 0.002 | 0.247 ± 0.00 [symbol] | 0.227 ± 0.00 [symbol] |
| Ours(no alignment) | 0.478 ± 0.005 | 0. [symbol] ± 0.004 | 0.33 [symbol] ± 0.003 | 0.271 ± 0.00 [symbol] | 0.2 [symbol] ± 0.004 |
| Ours(no augmentation) | 0. [symbol] ± 0.003 | 0. [symbol] ± 0.006 | 0.275 ± 0.004 | 0.22 [symbol] ± 0.007 | 0.211 ± 0.003 |
| Ours(no homology) | 0.47 [symbol] ± 0.003 | 0. [symbol] ± 0.007 | 0.341 ± 0.003 | 0.28 [symbol] ± 0.010 | 0.2 [symbol] 7 ± 0.004 |
| Ours(no [symbol] ) | 0. [symbol] ± 0.002 | 0. [symbol] ± 0.007 | 0.320 ± 0.005 | 0.289 ± 0.005 | 0.240 ± 0.00 [symbol] |

[symbol]

indicates data missing or illegible when filed

Referring to a result of measuring a mean of binding residues bound to the closest ligand by executing respective models of Table 1 above with respect respective databases, respectively, it can be seen that a prediction result for the binding site and the binding residue according to the exemplary embodiments of the present disclosure is more excellent than results in the existing models such as DeepSurf, Kalasanty, Deeppocket, etc.

TABLE 2
<Table showing measurement of a mean of binding residues compared to a detected ligand>

| | scPDB(held-out) | COACH420 | HOLO4k | CHEN-holo | CHEN- [symbol] |
|---|---|---|---|---|---|
| DeepSurf | 0.402 ± 0.010 | 0.419 ± 0.013 | 0.330 ± 0.007 | 0.372 ± 0.019 | 0.336 ± 0.020 |
| Ka [symbol] | 0.356 ± 0.000 | 0.3 [symbol] ± 0.000 | 0.344 ± 0.000 | 0.333 ± 0.000 | 0.323 ± 0.000 |
| Deeppocket | 0.595 ± 0.002 | 0.5 [symbol] ± 0.005 | 0.371 ± 0.004 | 0.395 ± 0.008 | 0.382 ± 0.000 |
| Ours | 0.643 ± 0.004 | 0.58 [symbol] ± 0.007 | 0.41 [symbol] ± 0.002 | 0.495 ± 0.010 | 0.473 ± 0.004 |
| Ours(BERT) | 0.5 [symbol] 7 ± 0.012 | 0.459 ± 0.013 | 0.356 ± 0.007 | 0. [symbol] ± 0.016 | 0.328 ± 0.010 |
| Ours(no CNN) | 0.624 ± 0.002 | 0.548 ± 0.004 | 0.395 ± 0.002 | 0.450 ± 0.014 | 0.419 ± 0.00 [symbol] |
| Ours(no alignment) | 0.637 ± 0.005 | 0.572 ± 0.001 | 0.413 ± 0.002 | 0. [symbol] ± 0. [symbol] | 0.45 [symbol] ± 0.00 [symbol] |
| Ours(no augmentation) | 0.522 ± 0.001 | 0.4 [symbol] ± 0.007 | 0.347 ± 0.004 | 0.397 ± 0.010 | 0.38 [symbol] ± 0.007 |
| Ours(no homology) | 0.628 ± 0.003 | 0.567 ± 0.009 | 0.412 ± 0.002 | 0.481 ± 0.007 | 0.44 [symbol] ± 0.010 |
| Ours(no [symbol] ) | 0. [symbol] ± 0.00 [symbol] | 0.547 ± 0.008 | 0.391 ± 0.004 | 0.408 ± 0.008 | 0.445 ± 0.008 |

[symbol]

indicates data missing or illegible when filed

Referring to a result of measuring a mean of binding residues compared to a detected ligand by executing respective models of Table 2 above with respect respective databases, respectively, it can be seen that the prediction result for the binding site and the binding residue according to the exemplary embodiments of the present disclosure is more excellent than results in the existing models such as DeepSurf, Kalasanty, Deeppocket, etc.

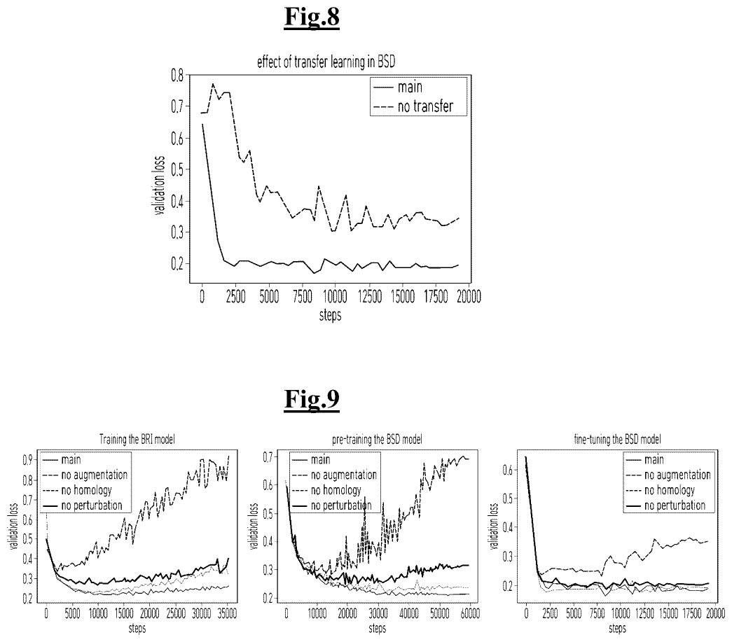

Meanwhile, FIG. 8 is a schematic view for comparing and describing performing transfer learning and not performing the transfer learning when training the first neural network model according to an exemplary embodiment of the present disclosure.

Referring to FIG. 8, a horizontal axis may mean training steps, and a vertical axis may mean a validation loss. In this case, a graph in a case of performing the transfer learning according to an exemplary embodiment of the present disclosure upon training the first neural network model is represented by a solid line and a graph in a case of not performing the transfer learning upon training the first neural network model is represented by dotted lines.

In this regard, when the solid line and the dotted lines of FIG. 8 are compared, it can be confirmed that in the case of performing the transfer learning for the first neural network model according to an exemplary embodiment of the present disclosure, a convergence speed significantly increases as compared with the case of not performing the transfer learning, and it can be seen that the validation loss also decreases to an almost converged leveling a 2500-th step. Additionally, according to the present disclosure, the validation loss in a convergence state is approximately 0.2 in the case of the solid line (the transfer learning is performed) and approximately 0.3 in the case of the dotted lines (the transfer learning is not performed) through the transfer learning, so it can be seen that the validation loss of the convergence state is remarkably improved through the transfer learning.

FIG. 9 is a schematic view for comparing and describing performing data augmentation and not performing the data augmentation when training the first and second neural network models according to an exemplary embodiment of the present disclosure.

Referring to FIG. 9, the horizontal axis may mean the training steps, and the vertical axis may mean the validation loss. Further,
1) according to an exemplary embodiment of the present disclosure, a case of performing the data augmentation through random conversion of the residue orientation is expressed as a graph of a thick solid line,
2) according to an exemplary embodiment of the present disclosure, a case of performing the data augmentation based on the protein homology is expressed as a graph of a tight dotted line,
3) according to an exemplary embodiment of the present disclosure, a case of performing all of various data augmentation processes (for example, the random conversion of the residue orientation and the augmentation based on the protein homology) is expressed as a graph of a normal solid line, and
4) a case where nothing is applied is expressed as a graph of a normal dotted line.

Through the graphs of FIG. 8, it can be seen that in the case of performing the data augmentation based on the protein homology or the data augmentation through the random conversion of the residue orientation according to an exemplary embodiment of the present disclosure, the performances of the first neural network model and the second neural network model are remarkably improved as compared with a case of not performing the data augmentation.

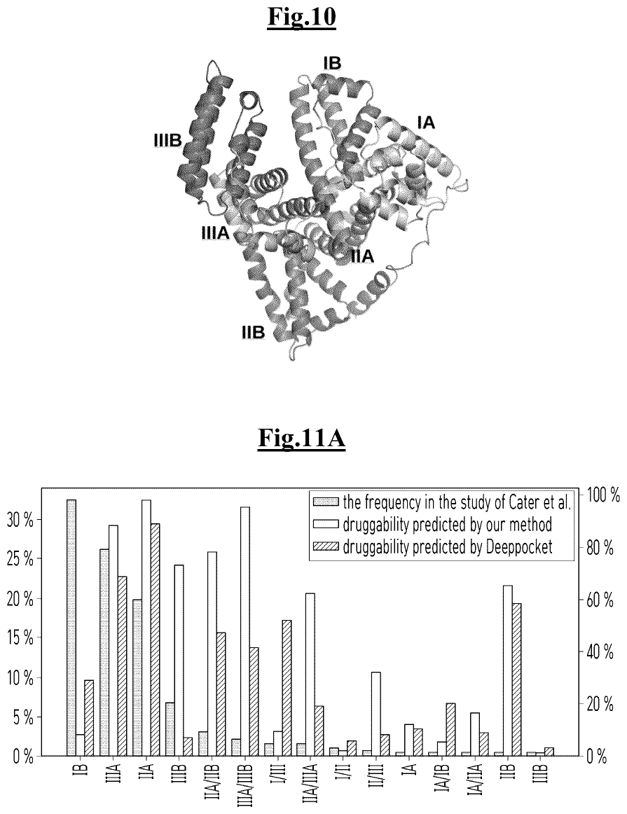

Meanwhile, FIGS. 9 to 11 are diagrams for describing a result of comparing respective existing models and results according to an exemplary embodiment of the present disclosure through a case study for human serum albumin.

In this regard, FIG. 9 is a schematic view illustrating a lower domain of human serum albumin (HSA) according to an exemplary embodiment of the present disclosure. First, referring to FIG. 9, the human serum albumin (HSA) may be constituted by three homologous domains I, II, and III, and each homologous domain may be constituted by two lower domains A and B.

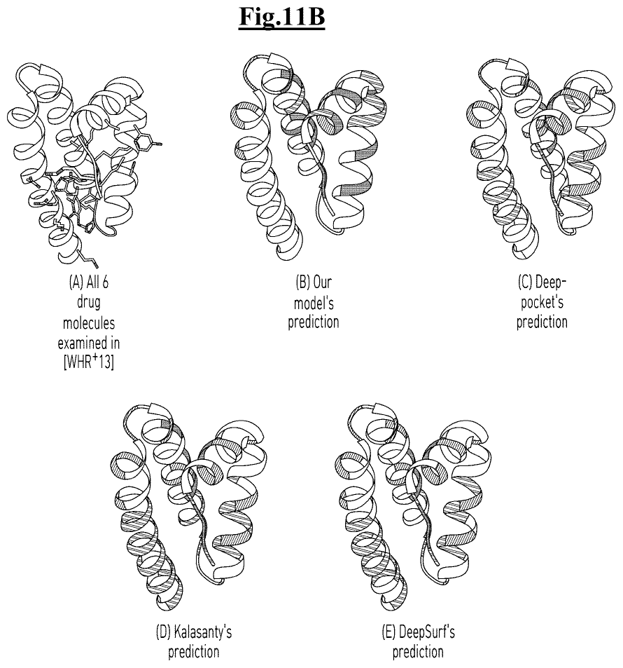

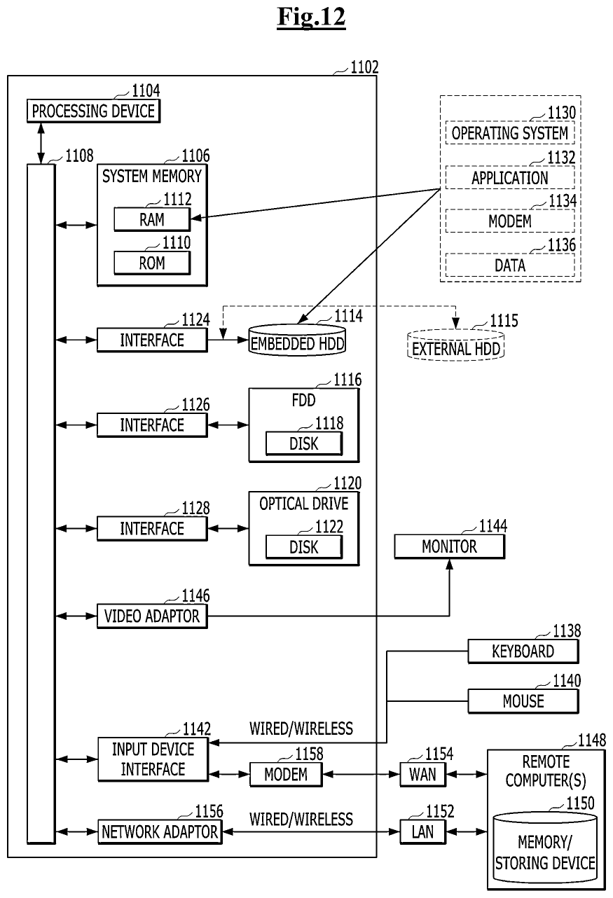

15 binding sites related to three homologous domains I, II, and III of the human serum albumin (HSA), and the respective lower domains A and B are predicted to evaluate the performance of the model, and hereinafter, a detailed description thereof will be described below through FIGS. 11A and 11B below.

FIGS. 11A and 11B are schematic views for comparing and describing a prediction result according to an exemplary embodiment of the present disclosure and a prediction result of existing methods.

In regard to the experimental result, referring to FIG. 11A, it can be confirmed that a prediction result according to an exemplary embodiment of the present disclosure is more excellent than the prediction result (for example, methods of cater and Deeppocket) of another model and study in terms of prediction results for 10 binding sites among 15 binding sites.

In regard to the experimental result, referring to FIG. 11B, a dark gray means a true-positive, and a slash pattern in which an interval is not constant and a slash in which the interval is constant mean false-positive and false-negative binding site residues, respectively. Accordingly, in the figure of FIG. 11B, it can be seen that the larger a dark gray area, the more true-positive prediction results, so the larger the dark gray area, the more excellent the prediction result for the binding site.

In this case, in FIG. 11B, it can be seen that when a prediction result B for the binding site according to an exemplary embodiment of the present disclosure is compared with experimental results A, and C to E of other models and studies, the dark gray area is large, so the prediction result for the binding site is more excellent than the prediction results for the binding sites of other models and studies.

Disclosed is a computer readable medium storing the data structure according to an exemplary embodiment of the present disclosure. The data structure may refer to the organization, management, and storage of data that enables efficient access to and modification of data. The data structure may refer to the organization of data for solving a specific problem (e.g., data search, data storage, data modification in the shortest time). The data structures may be defined as physical or logical relationships between data elements, designed to support specific data processing functions. The logical relationship between data elements may include a connection between data elements that the user defines. The physical relationship between data elements may include an actual relationship between data elements physically stored on a computer-readable storage medium (e.g., persistent storage device). The data structure may specifically include a set of data, a relationship between the data, a function which may be applied to the data, or instructions. Through an effectively designed data structure, a computing device performs operations while using the resources of the computing device to a minimum. Specifically, the computing device can increase the efficiency of operation, read, insert, delete, compare, exchange, and search through the effectively designed data structure.

The data structure may be divided into a linear data structure and a non-linear data structure according to the type of data structure. The linear data structure may be a structure in which only one data is connected after one data. The linear data structure may include a list, a stack, a queue, and a deque. The list may mean a series of data sets in which an order exists internally. The list may include a linked list. The linked list may be a data structure in which data is connected in a scheme in which each data is linked in a row with a pointer. In the linked list, the pointer may include link information with next or previous data. The linked list may be represented as a single linked list, a double linked list, or a circular linked list depending on the type. The stack may be a data listing structure with limited access to data. The stack may be a linear data structure that may process (e.g., insert or delete) data at only one end of the data structure. The data stored in the stack may be a data structure (LIFO-Last in First Out) in which the data is input last and output first. The queue is a data listing structure that may access data limitedly and unlike a stack, the queue may be a data structure (FIFO-First in First Out) in which late stored data is output late. The deque may be a data structure capable of processing data at both ends of the data structure.

The non-linear data structure may be a structure in which a plurality of data are connected after one data. The non-linear data structure may include a graph data structure. The graph data structure may be defined as a vertex and an edge, and the edge may include a line connecting two different vertices. The graph data structure may include a tree data structure. The tree data structure may be a data structure in which there is one path connecting two different vertices among a plurality of vertices included in the tree. That is, the tree data structure may be a data structure that does not form a loop in the graph data structure.

In the present disclosure, Operation model, neural network, a network function, and a neural network may be used to be exchangeable. From here on, it will be described uniformly using neural networks.

The data structure may include the neural network. In addition, the data structures, including the neural network, may be stored in a computer readable medium. The data structure including the neural network may also include data preprocessed for processing by the neural network, data input to the neural network, weights of the neural network, hyper parameters of the neural network, data obtained from the neural network, an active function associated with each node or layer of the neural network, and a loss function for training the neural network. The data structure including the neural network may include predetermined components of the components disclosed above. In other words, the data structure including the neural network may include all of data preprocessed for processing by the neural network, data input to the neural network, weights of the neural network, hyper parameters of the neural network, data obtained from the neural network, an active function associated with each node or layer of the neural network, and a loss function for training the neural network or a combination thereof. In addition to the above-described configurations, the data structure including the neural network may include predetermined other information that determines the characteristics of the neural network. In addition, the data structure may include all types of data used or generated in the calculation process of the neural network, and is not limited to the above. The computer readable medium may include a computer readable recording medium and/or a computer readable transmission medium. The neural network may be generally constituted by an aggregate of calculation units which are mutually connected to each other, which may be called nodes. The nodes may also be called neurons. The neural network is configured to include one or more nodes.

The data structure may include data input into the neural network. The data structure including the data input into the neural network may be stored in the computer readable medium. The data input to the neural network may include training data input in a neural network training process and/or input data input to a neural network in which training is completed. The data input to the neural network may include preprocessed data and/or data to be preprocessed. The preprocessing may include a data processing process for inputting data into the neural network. Therefore, the data structure may include data to be preprocessed and data generated by preprocessing. The data structure is just an example and the present disclosure is not limited thereto.

The data structure may include the weight of the neural network (in the present disclosure, the weight and the parameter may be used as the same meaning). In addition, the data structures, including the weight of the neural network, may be stored in the computer readable medium. The neural network may include a plurality of weights. The weight may be variable and the weight is variable by a user or an algorithm in order for the neural network to perform a desired function. For example, when one or more input nodes are mutually connected to one output node by the respective links, the output node may determine a data value output from an output node based on values input in the input nodes connected with the output node and the weights set in the links corresponding to the respective input nodes. The data structure is just an example and the present disclosure is not limited thereto.

As a non-limiting example, the weight may include a weight which varies in the neural network training process and/or a weight in which neural network training is completed. The weight which varies in the neural network training process may include a weight at a time when a training cycle starts and/or a weight that varies during the training cycle. The weight in which the neural network training is completed may include a weight in which the training cycle is completed. Accordingly, the data structure including the weight of the neural network may include a data structure including the weight which varies in the neural network training process and/or the weight in which neural network training is completed. Accordingly, the above-described weight and/or a combination of each weight are included in a data structure including a weight of a neural network. The data structure is just an example and the present disclosure is not limited thereto.

The data structure including the weight of the neural network may be stored in the computer-readable storage medium (e.g., memory, hard disk) after a serialization process. Serialization may be a process of storing data structures on the same or different computing devices and later reconfiguring the data structure and converting the data structure to a form that may be used. The computing device may serialize the data structure to send and receive data over the network. The data structure including the weight of the serialized neural network may be reconfigured in the same computing device or another computing device through deserialization. The data structure including the weight of the neural network is not limited to the serialization. Furthermore, the data structure including the weight of the neural network may include a data structure (for example, B-Tree, Trie, m-way search tree, AVL tree, and Red-Black Tree in a nonlinear data structure) to increase the efficiency of operation while using resources of the computing device to a minimum. The above-described matter is just an example and the present disclosure is not limited thereto.

The data structure may include hyper-parameters of the neural network. In addition, the data structures, including the hyper-parameters of the neural network, may be stored in the computer readable medium. The hyper-parameter may be a variable which may be varied by the user. The hyper-parameter may include, for example, a learning rate, a cost function, the number of training cycle iterations, weight initialization (for example, setting a range of weight values to be subjected to weight initialization), and Hidden Unit number (e.g., the number of hidden layers and the number of nodes in the hidden layer). The data structure is just an example and the present disclosure is not limited thereto.

FIG. 12 is a normal and schematic view of an exemplary computing environment in which the exemplary embodiments of the present disclosure may be implemented. It is described above that the present disclosure may be generally implemented by the computing device, but those skilled in the art will well know that the present disclosure may be implemented in association with a computer executable command which may be executed on one or more computers and/or in combination with other program modules and/or a combination of hardware and software.

In general, the program module includes a routine, a program, a component, a data structure, and the like that execute a specific task or implement a specific abstract data type. Further, it will be well appreciated by those skilled in the art that the method of the present disclosure can be implemented by other computer system configurations including a personal computer, a handheld computing device, microprocessor-based or programmable home appliances, and others (the respective devices may operate in connection with one or more associated devicesas well as a single-processor or multi-processor computer system, a mini computer, and a main frame computer.)

The exemplary embodiments described in the present disclosure may also be implemented in a distributed computing environment in which predetermined tasks are performed by remote processing devices connected through a communication network. In the distributed computing environment, the program module may be positioned in both local and remote memory storage devices.

The computer generally includes various computer readable media. Media accessible by the computer may be computer readable media regardless of types thereof and the computer readable media include volatile and non-volatile media, transitory and non-transitory media, and mobile and non-mobile media. As a non-limiting example, the computer readable media may include both computer readable storage media and computer readable transmission media. The computer readable storage media include volatile and non-volatile media, transitory and non-transitory media, and mobile and non-mobile media implemented by a predetermined method or technology for storing information such as a computer readable instruction, a data structure, a program module, or other data. The computer readable storage media include a RAM, a ROM, an EEPROM, a flash memory or other memory technologies, a CD-ROM, a digital video disk (DVD) or other optical disk storage devices, a magnetic cassette, a magnetic tape, a magnetic disk storage device or other magnetic storage devices or predetermined other media which may be accessed by the computer or may be used to store desired information, but are not limited thereto.

The computer readable transmission media generally implement the computer readable command, the data structure, the program module, or other data in a carrier wave or a modulated data signal such as other transport mechanism and include all information transfer media. The term “modulated data signal” means a signal acquired by setting or changing at least one of characteristics of the signal so as to encode information in the signal. As a non-limiting example, the computer readable transmission media include wired media such as a wired network or a direct-wired connection and wireless media such as acoustic, RF, infrared and other wireless media. A combination of any media among the aforementioned media is also included in a range of the computer readable transmission media.

An exemplary environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined processor among various commercial processors. A dual processor and other multi-processor architectures may also be used as the processing device 1104.

The system bus 1108 may be any one of several types of bus structures which may be additionally interconnected to a local bus using any one of a memory bus, a peripheral device bus, and various commercial bus architectures. The system memory 1106 includes a read only memory (ROM) 1110 and a random access memory (RAM) 1112. A basic input/output system (BIOS) is stored in the non-volatile memories 1110 including the ROM, the EPROM, the EEPROM, and the like and the BIOS includes a basic routine that assists in transmitting information among components in the computer 1102 at a time such as in-starting. The RAM 1112 may also include a high-speed RAM including a static RAM for caching data, and the like.

The computer 1102 also includes an interior hard disk drive (HDD) 1114 (for example, EIDE and SATA), in which the interior hard disk drive 1114 may also be configured for an exterior purpose in an appropriate chassis (not illustrated), a magnetic floppy disk drive (FDD) 1116 (for example, for reading from or writing in a mobile diskette 1118), and an optical disk drive 1120 (for example, for reading a CD-ROM disk 1122 or reading from or writing in other high-capacity optical media such as the DVD, and the like). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. An interface 1124 for implementing an exterior drive includes at least one of a universal serial bus (USB) and an IEEE 1394 interface technology or both of them.

The drives and the computer readable media associated therewith provide non-volatile storage of the data, the data structure, the computer executable instruction, and others. In the case of the computer 1102, the drives and the media correspond to storing of predetermined data in an appropriate digital format. In the description of the computer readable media, the mobile optical media such as the HDD, the mobile magnetic disk, and the CD or the DVD are mentioned, but it will be well appreciated by those skilled in the art that other types of media readable by the computer such as a zip drive, a magnetic cassette, a flash memory card, a cartridge, and others may also be used in an exemplary operating environment and further, the predetermined media may include computer executable commands for executing the methods of the present disclosure. Multiple program modules including an operating system 1130, one or more application programs 1132, other program module 1134, and program data 1136 may be stored in the drive and the RAM 1112. All or some of the operating system, the application, the module, and/or the data may also be cached in the RAM 1112. It will be well appreciated that the present disclosure may be implemented in operating systems which are commercially usable or a combination of the operating systems.

A user may input instructions and information in the computer 1102 through one or more wired/wireless input devices, for example, pointing devices such as a keyboard 1138 and a mouse 1140. Other input devices (not illustrated) may include a microphone, an IR remote controller, a joystick, a game pad, a stylus pen, a touch screen, and others. These and other input devices are often connected to the processing device 1104 through an input device interface 1142 connected to the system bus 1108, but may be connected by other interfaces including a parallel port, an IEEE 1394 serial port, a game port, a USB port, an IR interface, and others.

A monitor 1144 or other types of display devices are also connected to the system bus 1108 through interfaces such as a video adapter 1146, and the like. In addition to the monitor 1144, the computer generally includes other peripheral output devices (not illustrated) such as a speaker, a printer, others.

The computer 1102 may operate in a networked environment by using a logical connection to one or more remote computers including remote computer(s) 1148 through wired and/or wireless communication. The remote computer(s) 1148 may be a workstation, a computing device computer, a router, a personal computer, a portable computer, a micro-processor based entertainment apparatus, a peer device, or other general network nodes and generally includes multiple components or all of the components described with respect to the computer 1102, but only a memory storage device 1150 is illustrated for brief description. The illustrated logical connection includes a wired/wireless connection to a local area network (LAN) 1152 and/or a larger network, for example, a wide area network (WAN) 1154. The LAN and WAN networking environments are general environments in offices and companies and facilitate an enterprise-wide computer network such as Intranet, and all of them may be connected to a worldwide computer network, for example, the Internet.

When the computer 1102 is used in the LAN networking environment, the computer 1102 is connected to a local network 1152 through a wired and/or wireless communication network interface or an adapter 1156. The adapter 1156 may facilitate the wired or wireless communication to the LAN 1152 and the LAN 1152 also includes a wireless access point installed therein in order to communicate with the wireless adapter 1156. When the computer 1102 is used in the WAN networking environment, the computer 1102 may include a modem 1158 or has other means that configure communication through the WAN 1154 such as connection to a communication computing device on the WAN 1154 or connection through the Internet. The modem 1158 which may be an internal or external and wired or wireless device is connected to the system bus 1108 through the serial port interface 1142. In the networked environment, the program modules described with respect to the computer 1102 or some thereof may be stored in the remote memory/storage device 1150. It will be well known that an illustrated network connection is exemplary and other means configuring a communication link among computers may be used.

The computer 1102 performs an operation of communicating with predetermined wireless devices or entities which are disposed and operated by the wireless communication, for example, the printer, a scanner, a desktop and/or a portable computer, a portable data assistant (PDA), a communication satellite, predetermined equipment or place associated with a wireless detectable tag, and a telephone. This at least includes wireless fidelity (Wi-Fi) and Bluetooth wireless technology. Accordingly, communication may be a predefined structure like the network in the related art or just ad hoc communication between at least two devices.

The wireless fidelity (Wi-Fi) enables connection to the Internet, and the like without a wired cable. The Wi-Fi is a wireless technology such as the device, for example, a cellular phone which enables the computer to transmit and receive data indoors or outdoors, that is, anywhere in a communication range of a base station. The Wi-Fi network uses a wireless technology called IEEE 802.11(a, b, g, and others) in order to provide safe, reliable, and high-speed wireless connection. The Wi-Fi may be used to connect the computers to each other or the Internet and the wired network (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or operate in a product including both bands (dual bands).

It will be appreciated by those skilled in the art that information and signals may be expressed by using various different predetermined technologies and techniques. For example, data, instructions, commands, information, signals, bits, symbols, and chips which may be referred in the above description may be expressed by voltages, currents, electromagnetic waves, magnetic fields or particles, optical fields or particles, or predetermined combinations thereof.

It may be appreciated by those skilled in the art that various exemplary logical blocks, modules, processors, means, circuits, and algorithm steps described in association with the exemplary embodiments disclosed herein may be implemented by electronic hardware, various types of programs or design codes (for easy description, herein, designated as software), or a combination of all of them. In order to clearly describe the intercompatibility of the hardware and the software, various exemplary components, blocks, modules, circuits, and steps have been generally described above in association with functions thereof. Whether the functions are implemented as the hardware or software depends on design restrictions given to a specific application and an entire system. Those skilled in the art of the present disclosure may implement functions described by various methods with respect to each specific application, but it should not be interpreted that the implementation determination departs from the scope of the present disclosure.

Various exemplary embodiments presented herein may be implemented as manufactured articles using a method, a device, or a standard programming and/or engineering technique. The term manufactured article includes a computer program, a carrier, or a medium which is accessible by a predetermined computer-readable storage device. For example, a computer-readable storage medium includes a magnetic storage device (for example, a hard disk, a floppy disk, a magnetic strip, or the like), an optical disk (for example, a CD, a DVD, or the like), a smart card, and a flash memory device (for example, an EEPROM, a card, a stick, a key drive, or the like), but is not limited thereto. Further, various storage media presented herein include one or more devices and/or other machine-readable media for storing information.

It will be appreciated that a specific order or a hierarchical structure of steps in the presented processes is one example of exemplary accesses. It will be appreciated that the specific order or the hierarchical structure of the steps in the processes within the scope of the present disclosure may be rearranged based on design priorities. Appended method claims provide elements of various steps in a sample order, but the method claims are not limited to the presented specific order or hierarchical structure.

The description of the presented exemplary embodiments is provided so that those skilled in the art of the present disclosure use or implement the present disclosure. Various modifications of the exemplary embodiments will be apparent to those skilled in the art and general principles defined herein can be applied to other exemplary embodiments without departing from the scope of the present disclosure. Therefore, the present disclosure is not limited to the exemplary embodiments presented herein, but should be interpreted within the widest range which is coherent with the principles and new features presented herein.

1. A method for predicting a binding site of a protein, the method performed by a computing device, the method comprising:
obtaining one or more candidate data;
filtering the one or more candidate data, and obtaining the filtered candidate data, by using a first neural network model for detecting a binding site; and
predicting a binding residue based on the filtered candidate data by using a second neural network model for identifying the binding residue,
wherein the first neural network model shares some parameters with the second neural network model.

2. The method of claim 1, wherein the one or more candidate data includes:
one or more candidate binding sites of a protein, or a center of at least one of the candidate binding sites, and
wherein the obtaining the one or more candidate data includes:
obtaining the one or more candidate binding sites of a protein, or the center of at least one of the candidate binding sites, by using an algorithm for predicting the binding site in a protein structure.

3. The method of claim 1, wherein the first neural network model includes:
a first sub neural network for extracting a local feature of the binding site; and
a second sub neural network for globally aggregating the local features, and
wherein the second neural network model shares at least one of the first sub neural network or the second sub neural network with the first neural network model.

4. The method of claim 3, wherein the first sub neural network for extracting the local feature of the binding site includes a 3D convolutional network,
wherein the second sub neural network for globally aggregating the local features includes a geometric attention layer, and
wherein the first neural network model further includes a third sub neural network for mapping the feature aggregated through the second sub neural network to a single scalar quantity.

5. The method of claim 3, wherein the first sub neural network performs an operation of applying a grid alignment.

6. The method of claim 3, wherein the second sub neural network performs an operation of randomly transforming an orientation of a residue in a training process of at least one of the first neural network or the second neural network.

7. The method of claim 1, wherein the filtering the one or more candidate data, and obtaining the filtered candidate data, by using the first neural network model for detecting a binding site includes:
calculating a score for each of the one or more candidate data by using the first neural network model for detecting the binding site; and
obtaining the filtered candidate data based on the score for each of the candidate data.

8. The method of claim 1, wherein the first neural network model is a model trained based on:
an operation of obtaining first training data and first ground truth data corresponding to the first training data;
an operation of predicting a training binding site based on the first training data using the first neural network model; and
an operation of training the first neural network model based on the predicted training binding site and the first ground truth data, and
wherein the second neural network model is a model trained based on:
an operation of obtaining second training data and second ground truth data corresponding to the second training data;
an operation of predicting a training binding residue based on the second training data using the second neural network model; and
an operation of training the second neural network model based on the predicted training binding residue and the second ground truth data.

9. The method of claim 8, wherein the first neural network model is a model trained based on an operation of performing training for the first neural network model after performing a training process of the second neural network model.

10. The method of claim 9, wherein the first neural network model shares some parameters with the trained second neural network model,
wherein the operation of performing training for the first neural network model after performing the training process of the second neural network model includes:
an operation of setting some parameters shared with the trained second neural network model as an initial condition, and
an operation of performing the training for the first neural network model based on the set initial condition.

11. A method for predicting a binding site of a protein, the method performed by a computing device, the method comprising:
obtaining training data, ground truth data corresponding to the training data, and external data;
aligning the training data and the external data, and obtaining aligned external data corresponding to the training data; and
assigning first sub data of the training data as sub data of the aligned external data based on the ground truth data to obtain augmented data,
wherein the augmented data is used in a process of training at least one of a first neural network model for detecting the binding site or a second neural network model for identifying the binding residue.

12. The method of claim 11, wherein the aligning the training data and the external data, and obtaining the aligned external data corresponding to the training data includes:
obtaining an amino acid sequence associated with the training data; and
when the obtained amino acid sequence is conserved in the aligned external data with the training data, obtaining aligned external data corresponding to the training data.

13. The method of claim 11, wherein the training data includes a training protein structure, and
wherein the ground truth data includes one or more ground truth binding sites for the training protein structure and a center of each of the ground truth binding sites, and
wherein the assigning the first sub data of the training data as the sub data of the aligned external data based on the ground truth data to obtain the augmented data includes:
assigning the center of the ground truth binding site to a center of a binding site included in the aligned external data to obtain first augmented data.

14. The method of claim 13, wherein the assigning the center of the ground truth binding site to the center of the binding site corresponding to the training data included in the aligned external data to obtain the first augmented data includes:
obtaining a center of a candidate binding site by using an algorithm for predicting a binding site in a protein structure for the external data;
obtaining a center of the binding site of the aligned external data corresponding to the training data;
measuring a distance between the obtained center of the candidate binding site of the aligned external data and the center of the binding site corresponding to the training data; and
when the measured distance is within a predetermined threshold, assigning the center of the ground truth binding site to the center of the binding site of the aligned external data corresponding to the training data to obtain the first augmented data.

15. The method of claim 13, wherein the ground truth data further includes a ground truth binding residue for each of the ground truth binding sites, and
the assigning the center of the ground truth binding site to the center of the binding site of the aligned external data corresponding to the training data to obtain the first augmented data includes:
calculating a ratio at which the binding residue of the aligned external data corresponding to the training data corresponds to the ground truth binding residue; and
when the calculated ratio is equal to or more than a predetermined threshold, assigning the center of the ground truth binding site to the center of the binding site of the aligned external data corresponding to the training data to obtain the first augmented data.

16. The method of claim 11, wherein the training data includes a first training protein structure,
wherein the ground truth data includes a first ground truth binding residue for the first training protein structure, and
wherein the assigning the first sub data of the training data as the sub data of the aligned external data based on the ground truth data to obtain the augmented data includes:
assigning the first ground truth binding residue to the binding residue of the aligned external data corresponding to the training data to obtain second augmented data.

17. The method of claim 16, wherein the training data further includes a second training protein structure,
wherein the ground truth data includes a second ground truth biding residue for the second training protein structure, and
wherein the assigning the first ground truth binding residue to the binding residue of the aligned external data corresponding to the training data to obtain the second augmented data includes:
assigning the first ground truth binding residue to a first binding residue of the aligned external data corresponding to the training data to obtain a first assigned binding residue;
assigning the second ground truth binding residue to a second binding residue of the aligned external data corresponding to the training data to obtain a second assigned binding residue; and
obtaining the second augmented data based on the first assigned binding residue and the second assigned binding residue.

18. A computer program stored in a computer-readable storage medium, wherein the computer program causes one or more processors to perform operations for predicting a binding site of a protein when the computer program is executed by the one or more processors, the operations comprising:
an operation of obtaining one or more candidate data;
an operation of filtering the one or more candidate data, and obtaining the filtered candidate data, by using a first neural network model for detecting a binding site; and
an operation of predicting a binding residue based on the filtered candidate data by using a second neural network model for identifying the binding residue,
wherein the first neural network model shares some parameters with the second neural network model.

19. The computer program of claim 18, wherein the operation of filtering the one or more candidate data, and obtaining the filtered candidate data, by using the first neural network model for detecting a binding site includes:
an operation of calculating a score for each of the one or more candidate data by using the first neural network model for detecting the binding site; and
an operation of obtaining the filtered candidate data based on the score for each of the candidate data.

20. (canceled)

21. (canceled)
